(19) United States
(12) Patent Application Publication
(10) Pub. No.: US 2009/0208488 A1
(43) Pub. Date: Aug. 20, 2009

(54) Regulation of dendritic cell functions by the DCAL-2 receptor

(75) Inventors: Edward A. Clark, Seattle, WA (US); Chang Hung Chen, Seattle, WA (US); Helen Floyd, York (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The University of Washington, Seattle, WA (US)

(21) Appl. No.: 11/805,350

(22) Filed: May 22, 2007

Related U.S. Application Data
(62) Division of application No. 11/048,610, filed on Jan. 31, 2005, now abandoned.

(51) Int. Cl. A61K 39/395; A61K 35/12; A61P 37/00

(52) U.S. Cl. 424/133.1; 424/139.1; 424/93.7

(57) ABSTRACT

This invention provides antibodies that specifically bind to DCAL-2 and other DCAL-2 reagents that modulate dendritic cell function. Modulators of the receptor, including modulators that alter DCAL-2 associated signals to and from DCs, can be used to alter dendritic cell function and to enhance or inhibit immune responses to cancer antigens, autoantigens, or pathogens.

### CROSS-REFERENCE TO RELATED APPLICATIONS

This application is a divisional of U.S. application Ser. No. 11/048,610, filed Jan. 31, 2005, which application is herein incorporated by reference.

### STATEMENT AS TO RIGHTS TO INVENTIONS MADE UNDER FEDERALLY SPONSORED RESEARCH AND DEVELOPMENT

This invention was made with Government support under Grant No. A144257, AI52203, No. RR00166, and Nos. DE13061 and DE13325, awarded by the National Institutes of Health. The Government has certain rights in this invention.

### BACKGROUND OF THE INVENTION

Dendritic cells (DCs) play a key role in the immune system in presenting antigens for initiating primary immune responses and T-cell-mediated immune responses. DCs have widespread tissue distribution and are generally present in the body at locations that are routinely exposed to foreign antigens, such as the skin, lung, gut, blood and lymphoid tissues.

DCs express various surface receptors. These include members of the C-type lectin (CLR) family, some of which bind sugars in a calcium-dependent manner using highly conserved carbohydrate recognition domains (CRDs). C-type lectin-like receptors are also present on other cells. For example, Marshall et al. (J. Biol. Chem. 278:14702-14802, 2004) describe that DCAL-2 receptors are present on granulocytes and monocytes, but contain no teachings that suggest that the receptors are present on dendritic cells. Bakker et al. (Cancer Res. 64:8443-8450, 1994) describe a protein that is almost identical to the DCAL-2 of SEQ ID NO:1, but has an additional 10 amino acids at the N-terminus, which is expressed on acute myeloid leukemia cells. However, there has been no teaching or suggestion in the art that DCAL-2 is involved in dendritic cell function or that antibodies to DCAL-2 can influence immune responses stimulated by DCs.

The current invention is thus based on the discovery the DCAL-2 receptor is expressed on the surface of dendritic cells and that DCAL-2 plays a role in dendritic cell function.

### BRIEF SUMMARY OF THE INVENTION

The current invention provides antibodies that specifically bind to DCAL-2 and other DCAL-2 reagents that modulate dendritic cell function. Modulators of the receptor, including modulators that alter DCAL-2 associated signals to and from DCs, can be used to alter dendritic cell functions and to enhance or inhibit immune responses to cancer antigens, autoantigens, or pathogens. For example, modulators can be used to influence DC maturation and to alter T-cell-mediated immune responses. Dendritic cells can be treated either in vitro or in vivo with a modulator, e.g., an antibody.

Thus, in one aspect, the invention provides a monoclonal antibody that specifically binds to a polypeptide comprising the sequence set forth in SEQ ID NO:1, wherein the antibody modulates dendritic cell function. The dendritic cell function can be any number of functions, including the ability to initiate a T-cell response; the ability to modulate dendritic cell response to pathogens; and the ability to increase dendritic cell cytokine and/or chemokine expression. Examples of the antibody-induced expression pattern changes in dendritic cells include expression of cytokines and/or chemokines such as α-interferon, TNFα, IL-12, IL-10, IL-6 and MIP3β.

In some embodiments, the antibody is an antibody that binds to the same epitope as the monoclonal antibody UW70. Such an antibody can be UW70, or a humanized version thereof.

Antibodies of the invention that specifically bind to DCAL-2, e.g., SEQ ID NO:1 and alter dendritic cell function are often recombinantly produced monoclonal antibodies. In some embodiments, the antibodies are binding fragments, e.g., FV fragments; engineered antibodies, e.g., humanized antibodies or chimeric antibodies; human antibodies; and the like. Dendritic cells that can be modulated by a DCAL-2 antibody of the invention include myeloid and mucosal dendritic cells.

In another aspect, the invention provides a method of screening for a modulator of DCAL-2 activity, the method comprising: contacting a candidate agent with a DCAL-2 polypeptide comprising the extracellular domain of SEQ ID NO:1; determining whether the candidate agent binds the DCAL-2 polypeptide; determining whether the candidate agent modulates dendritic cell function; and selecting a compound that binds to the DCAL-2 polypeptide and modulates dendritic cell function. In some embodiments, the step of determining whether the candidate agent modulates dendritic cell function comprises detecting the ability of the candidate agent to initiate a T-cell response; detecting the ability of the candidate agent to modulate the response to a pathogen; and detecting an increase in expression of a cytokine and/or a chemokine in dendritic cells. Such cytokines or chemokines include at least one of α-interferon, TNFα, IL-12, IL-10, IL-6 and MIP3β. IN other

The DCAL-2 polypeptide used in the screening methods is often recombinant. Candidate compounds or agents that are screen include antibody molecules, small molecules, and peptides, including soluble DCAL-2 receptor peptides.

In one embodiment, the step of determining whether the candidate agent binds to the DCAL-2 polypeptide comprises a competition assay using an antibody that specifically binds to the DCAL-2 sequence set forth in SEQ ID NO:1.

In another aspect, the invention provides a pharmaceutically acceptable carrier and a monoclonal antibody of the invention. In some embodiments, the antibody is an antibody that binds to the same epitope as the monoclonal antibody UW70. Such an antibody can be UW70, or a humanized version thereof. Antibodies of the invention to be used in pharmaceutical compositions are typically recombinantly produced. Antibodies of the invention include binding fragments, e.g., FV fragments; engineered antibodies, e.g., humanized antibodies of chimeric antibodies; human antibodies; and the like.

In another aspect, the invention provides a method of enhancing an immune response in a subject that has cancer, the method comprising administering a DCAL-2 agonist to the subject in an amount sufficient to enhance an immune response. The DCAL-2 agonist can be, e.g., an antibody, a small molecule, or a peptide agonist. The DCAL-2 agonist is typically an antibody that alters dendritic cell function. In some embodiments, such an antibody enhances immune responses to cancer antigens. Where the subject is human, the antibody is typically humanized or human. The antibody can be, e.g., a binding fragment, such as an scFV.

In some embodiments, the method further comprises administering a cancer vaccine. The cancer vaccine can comprise a polypeptide cancer antigen or a nucleic acid-based vaccine that encodes an antigen associated with cancer.

In another aspect, the invention provides a method of enhancing an immune response in a subject that has cancer, the method comprising treating dendritic cells ex vivo with a DCAL-2 agonist, e.g., a DCAL-2 antibody that specifically binds to SEQ ID NO:1 and increases dendritic cell-mediated T cell responses, in an amount sufficient to stimulate dendritic cell activity; and introducing the treated dendritic cells into the subject. Treatment with the DCAL-2 agonist can also further comprise administering a cancer antigen to the dendritic cells.

In another aspect, the invention provides a method of modulating an immune response in a subject, the method comprising administering an agent that blocks DCAL-2 dendritic cell functions that enhance immune responses, e.g., dendritic cell functions that are induced by a DCAL-2 antibody agonist as described herein. The agent can be any agent that blocks DCAL-2 function, including a soluble DCAL-2, a small molecule, or a peptide that blocks DCAL-2 signaling events. The agent is often a monoclonal antibody, such as a humanized or human antibody, where the subject is human. In other embodiments, the antibody is a soluble DCAL-2. The subject can have an autoimmune disease selected from the group consisting of multiple sclerosis, psoraisis, rheumatoid arthritis, and insulin-dependent diabetes. In some embodiments, the subject can have asthma, an allergy or a chronic inflammatory disease. In still other embodiments, the agent may be used to treat a subject to reduce transplantation reactions for allogenic or xenogenic transplants, or graft versus host disease in bone marrow transplants.

The invention also provides a pharmaceutical composition comprising a soluble DCAL-2 receptor and a pharmaceutically acceptable carrier.

In another aspect, the invention provides a method of augmenting a protective immune response, the method comprising administering an administering a monoclonal antibody that specifically binds to a polypeptide comprising the sequence set forth in SEQ ID NO:1, wherein the antibody modulates dendritic cell function; with a vaccine to a cancer antigen or pathogen, where the antibody is administered in an amount sufficient to augment the response to the antigen. The vaccine and the monoclonal antibody need not be administered at the same, time, but can be administered consecutively, in any order. The pathogen can be a viral pathogen, a bacterial pathogen, a fungal pathogen, or any other infectious agent for which it is desired to enhance immune response.

### BRIEF DESCRIPTION OF THE DRAWINGS

FIG. 1A-1C shows a protein-encoding cDNA sequence (SEQ ID NO:2) and polypeptide sequence (SEQ ID NO:1) of human DCAL-2 (FIG. 1A), the structure of the gene (FIG. 1B), and a phylogenetic tree of similar C-type lectins (FIG. 1C).

FIG. 2 shows a mouse DCAL-2 nucleic acid (SEQ ID NO:3) and protein (SEQ ID NO:4) sequence. Conserved regions corresponding to the amino acid residues noted in FIG. 1A are indicated in the mouse protein sequence.

FIG. 3A-3D provides exemplary data showing mRNA expression of DCAL-2 in various human tissues and cell lines.

FIGS. 4A and 4B provides exemplary data showing protein expression determined using monoclonal antibody UW70.

FIG. 5 provides exemplary data showing that tyrosine phosphorylation is induced upon cross-linking of DCAl-2 with mAb.

FIGS. 6A and 6B provide exemplary data showing that DCAl-2 is internalized after ligand binding.

FIG. 7 provides exemplary data showing that DCAl-2 interacts with different Toll-like receptors (TLRs) during DC maturation and augments DC maturations processes.

FIGS. 8a-8e provide data showing that anti-DCAl-2 mAB induces changes of DC cytokine expression profiles and that DCAl-2 signaling induces cytokine expression changes.

FIGS. 9a-9c provide exemplary data showing that DCAL-2 signaling synergizes with CD40 signals to promote inflammatory cytokine expression.

FIGS. 10a and 10b provide exemplary data showing that DCAL-2 signaling modulates the capacity of DCs to induce T cell activities.

### DETAILED DESCRIPTION OF THE INVENTION

### Definitions

The term “DCAL-2” refers to polypeptide polymorphic variants, alleles, mutants, and interspecies homologs and domains thereof that: (1) have an amino acid sequence that has greater than about 65% amino acid sequence identity, 70%, 75%, 80%, 85%, 90%, preferably 91%, 92%, 93%, 94%, 95%, 96%, 97%, 98% or 99% or greater amino acid sequence identity, preferably over a window of at least about 25, 50, 100, 200, 500, 1000, or more amino acids, to a sequence of SEQ ID NO:1; (2) bind to antibodies raised against an immunogen comprising an amino acid sequence of SEQ ID NO:1 and conservatively modified variants thereof; or (3) have at least 15 contiguous amino acids, more often, at least 20, 30, 40, 50, 100, 200, or 250, contiguous amino acids of SEQ ID NO:1. This term also refers to a domain of a DCAL-2 polypeptide, or a fusion protein comprising a domain of a DCAL-2 polypeptide linked to a heterologous protein. A DCAL-2 polypeptide can be either naturally occurring or recombinant.

A “full length” DCAL-2 protein or nucleic acid refers to a polypeptide or polynucleotide sequence, or a variant thereof, that contains all of the elements normally contained in one or more naturally occurring, wild type DCAL-2 polynucleotide or polypeptide sequences. It will be recognized, however, that derivatives, homologs, and fragments of DCAL-2 can be readily used in the present invention.

A “DCAL-2 modulator” or “DCAL-2 reagent” as used herein refers to an agent that binds to DCAL-2 and modulates dendritic cell function. Dendritic cell functions that are altered in response to DCAL-2 binding agents are “DCAL-2-associated” dendritic cell functions in the context of this application. A DCAL-2 modulator thus can also modulate signaling received by or sent by a dendritic cell. For the purposes of the patent application, a DCAL-2 “agonist” induces cytokines in DCs and enhances DC-mediated T-cell responses.

A “DCAL-2 soluble receptor” as used herein refers to a DCAL-2 polypeptide that is not bound to a cell membrane. Thus, a DCAL-2 soluble receptor is typically an extracellular domain and lacks transmembrane and cytoplasmic domains.

A “dendritic cell” as used herein refers to a bone-marrow derived cell that can internalize antigen and process the antigen, such that the antigen, or peptide derived from the antigen, is presented in the context of both MHC class I complex and the MHC class II complexes. A dendritic cell of the invention typically has the phenotype and characteristics of the DCs described in Steinman et al., Annual Rev. Immunol. 9:271-296, 1991 and in Banchereau and Steinman Nature 392:245-252, 1998. Dendritic cells include both immunogenic and tolerogenic antigen presenting cells, and may be classified as immature, semi-mature, or fully mature.

As used herein, the term “immature dendritic cells” refers to dendritic cells that lack the cell surface markers found on mature DCs, such as CD83, and CD14; express low levels of CCR7 and the cytosolic protein DC-LAMP, and low levels of the costimulatory molecules CD40, CD80 and CD 86, and usually express CD1a and CCR1, CCR2, CCR5 and CXCR1.

As used herein, the term “mature dendritic cells” refers to DCs that have increased expression of MHC class II, CD40, CD80, CD83 and CD86 as well as DC-LAMP; are characterized by their release of proinflammatory cytokines, and their ability to cause increased proliferation of naïve allogeneic T cells and/or increased production of DC cytokines in a mixed lymphocyte reaction. Mature DCs typically express high levels of CCR7, and CXCR4 and low levels of CCR1 and CCR5.

As used herein, the term “semi-mature dendritic cells” refers to DCs that have lost some of the characteristics of immature DCs but do not have all the characteristics of a mature DC phenotype and are characterized by their ability to induce a tolerogenic immune response to self-antigens.

As used herein, “antibody” includes reference to an immunoglobulin molecule immunologically reactive with a particular antigen, and includes both polyclonal and monoclonal antibodies. Various isotypes of antibodies exist, for example IgG1, IgG2, IgG3, IgG4, and other Ig, e.g., IgM, IgA, IgE isotypes. The term also includes genetically engineered forms such as chimeric antibodies (e.g., humanized murine antibodies) and heteroconjugate antibodies (e.g., bispecific antibodies). The term “antibody” includes fragments with antigen-binding capability (e.g., Fab′, F(ab′)₂, Fab, Fv and rIgG. See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301. Various antigen binding domain-fusion proteins are also disclosed, e.g., in US patent application Nos. 2003/0118592 and 2003/0133939, and are encompassed within the term “antibody” as used in this application.

An antibody immunologically reactive with a particular antigen can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs has been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” or a “VH” refer to the variable region of an immunoglobulin heavy chain of an antibody, including the heavy chain of an Fv, scFv, or Fab. References to “V_{L}” or a “VL” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody can also comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background.

Specific binding to an antibody under such conditions requires an antibody that is selected for its specificity for a particular protein. For example, polyclonal antibodies raised to a particular protein, polymorphic variants, alleles, orthologs, and conservatively modified variants, or splice variants, or portions thereof, can be selected to obtain only those polyclonal antibodies that are specifically immunoreactive with DCAL-2 proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies: A Laboratory Manual (1988) and Harlow & Lane, Using Antibodies (1999) for a description of immunoassay formats and conditions that can be used to determine specific immunoreactivity).

### Introduction

This invention provides modulators of dendritic cell function that exert their effects through DCAL-2. The modulators are often antibodies or DCAL-2 polypeptides, e.g., soluble receptors. Such modulators can be used to modulate the immune response. Further, DCAL-2-mediated alterations in dendritic cell function can be used in assays to identify modulators of DCAL-2 activities.

### DCAL-2 Polypeptide Sequences

DCAL-2 polypeptide sequences that can be used in the practice of this invention are known in the art (see, e.g., Marshall et al., J. Biol. Chem. 279:14792-14802, 2004). Exemplary human and mouse DCAL-2 nucleic acid sequences and their protein translates are available under accession numbers AY498550, AY547296, NM_{—}138337, and NM_{—}177686. Exemplary mouse and human DCAL-2 polypeptide sequence are available under accession numbers AAS00605, AAT11783, and NP_{—}808354.

DCAL-2 polypeptides can be used in screening assays, for example to identify DCAL-2 modulators, and to generate blocking agents, e.g., antibodies or DCAl-2 polypeptides such as soluble receptors, or agonist modulators of DCAL-2-mediated dendritic cell functional effects. In some embodiments, the extracellular domain of DCAL-2 is used as an immunogen to obtain monoclonal antibodies to DCAL-2.

Variants of DCAL-2 polypeptides can also be used. Such variants typically have at least 70% identity, preferably at least 75%, 80%, 85%, 90%, or 95% identity, to a region of at least 25 amino acids, typically 50 amino acids, or to the full length DCAL-2 polypeptide set forth in SEQ ID NO:1.

Percent identity can be used using well known algorithms or can be determined by manual alignment. Optimal alignment of sequences for comparison can be conducted, e.g., by the local alignment algorithm of Smith & Waterman, Adv. Appl. Math. 2:482 (1981), by the global alignment algorithm of Needleman & Wunsch, J. Mol. Biol. 48:443 (1970), by the search for similarity method of Pearson & Lipman, Proc. Nat.'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). Other examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Blast and the Smith & Waterman alignment with the default parameters are often used when comparing sequences as described herein.

DCAL-2 polypeptides for use in the invention are conveniently produced recombinantly using known techniques (see, e.g., Sambrook & Russell, Molecular Cloning, A Laboratory Manual (3rd Ed, 2001); Kriegler, Gene Transfer and Expression: A Laboratory Manual (1990); and Current Protocols in Molecular Biology (Ausubel et al., eds., 1994-1999)). Alternatively, they can be purified from natural sources.

### Antibodies to DCAL-2

The invention provides methods of modulating dendritic cell function, typically using DCAL-2 binding agents. In some embodiments of the invention, antibodies are used as modulators, either through the ability to bind to DCAL-2 and transduce a signal, i.e., as agonists and/or by blocking DCAL-2-ligand binding interactions.

Methods of preparing polyclonal and monoclonal antibodies are known to the skilled artisan (e.g., Coligan, supra; and Harlow & Lane, supra). Polyclonal antibodies can be raised in an animal, e.g., by one or more injections into a mammal of an immunizing agent and, if desired, an adjuvant.

Antibodies for use in the invention are typically monoclonal antibodies. Monoclonal antibodies may be prepared using hybridoma methods, which are well know in the art (see, e.g., Harlow and Lane, supra). In a hybridoma method, a mouse or other appropriate host animal, is typically immunized with an immunizing agent to elicit lymphocytes that produce or are capable of producing antibodies that specifically bind to the immunizing agent. Alternatively, the lymphocytes may be immunized in vitro. For this invention, the immunizing agent includes a DCAL-2 polypeptide, e.g., SEQ ID NO:1, or a fragment or fusion protein thereof.

In some embodiments, a monoclonal antibody that binds the same epitope as the monoclonal antibody described in the examples is used. The ability of a particular antibody to recognize the same epitope as another antibody is typically determined by the ability of one antibody to competitively inhibit binding of the second antibody to the antigen. Any of a number of competitive binding assays can be used to measure competition between two antibodies to the same antigen. For example, a sandwich ELISA assay can be used for this purpose. This is carried out by using a capture antibody to coat the surface of a well. A subsaturating concentration of tagged-antigen is then added to the capture surface. This protein will be bound to the antibody through a specific antibody:epitope interaction. After washing a second antibody, which has been covalently linked to a detectable moiety (e.g., HRP, with the labeled antibody being defined as the detection antibody) is added to the ELISA. If this antibody recognizes the same epitope as the capture antibody it will be unable to bind to the target protein as that particular epitope will no longer be available for binding. If however this second antibody recognizes a different epitope on the target protein it will be able to bind and this binding can be detected by quantifying the level of activity (and hence antibody bound) using a relevant substrate. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

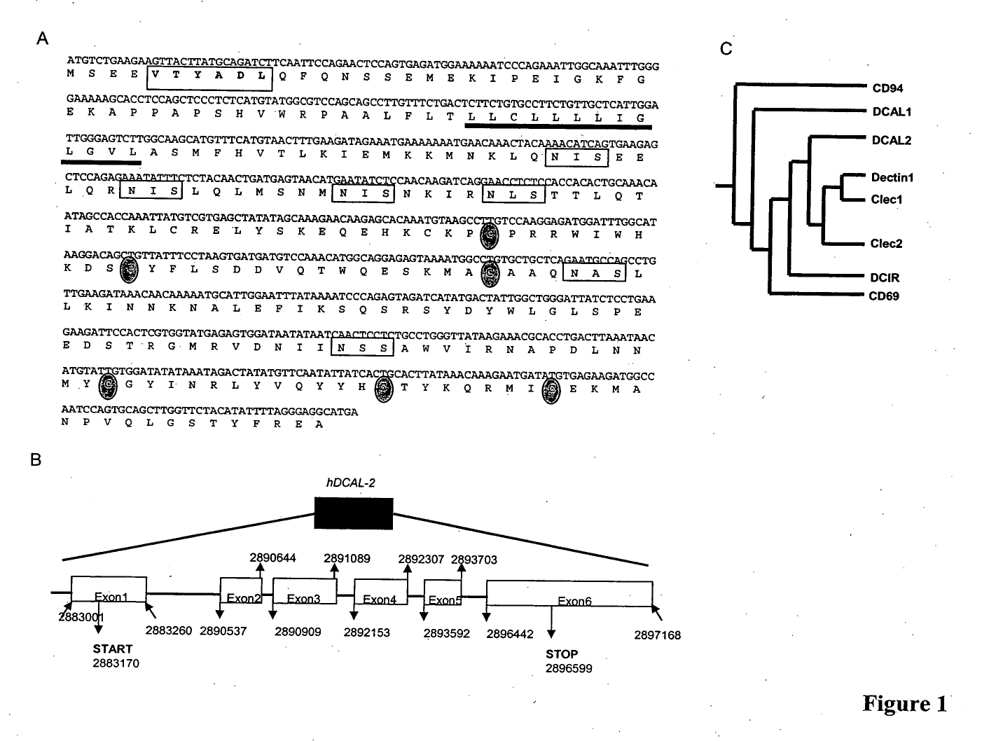

In preferred embodiments, a monoclonal anti-DCAL-2 antibody of the invention binds to the extracellular domain of SEQ ID NO:1 (amino acid residues 61 through the C-terminal end of SEQ ID NO:1). The black underlined section in FIG. 1A is from residues 48-60 but the transmembrane domain may extend to residue 44 since the LFLT (SEQ ID NO:5) sequence is hydrophobic.

In some embodiments the antibodies to the DCAL-2 protein are chimeric or humanized antibodies. As noted above, humanized forms of antibodies are chimeric immunoglobulins in which residues from a complementary determining region (CDR) of human antibody are replaced by residues from a CDR of a non-human species such as mouse, rat or rabbit having the desired specificity, affinity and capacity.

Antibodies can also be human antibodies. Human antibodies can be produced using various techniques known in the art, including phage display libraries (Hoogenboom & Winter, J. Mol. Biol. 227:381 (1991); Marks et al., J. Mol. Biol. 222:581 (1991)). Other techniques are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

In some embodiments, the antibody is a single chain Fv (scFv). The V_{H} and the V_{L} regions of a scFv antibody comprise a single chain which is folded to create an antigen binding site similar to that found in two chain antibodies. Once folded, noncovalent interactions stabilize the single chain antibody. While the V_{H} and V_{L} regions of some antibody embodiments can be directly joined together, one of skill will appreciate that the regions may be separated by a peptide linker consisting of one or more amino acids. Peptide linkers and their use are well-known in the art. See, e.g., Huston et al., Proc. Nat'l Acad. Sci. USA 8:5879 (1988); Bird et al., Science 242:4236 (1988); Glockshuber et al., Biochemistry 29:1362 (1990); U.S. Pat. No. 4,946,778, U.S. Pat. No. 5,132,405 and Stemmer et al., Biotechniques 14:256-265 (1993). Generally the peptide linker will have no specific biological activity other than to join the regions or to preserve some minimum distance or other spatial relationship between the V_{H} and V_{L}. However, the constituent amino acids of the peptide linker may be selected to influence some property of the molecule such as the folding, net charge, or hydrophobicity. Single chain Fv (scFv) antibodies optionally include a peptide linker.

Methods of making scFv antibodies have been described. See, Huse et al., supra; Ward et al. supra; and Vaughan et al., supra.

In some embodiments, the antibodies are bispecific antibodies. Bispecific antibodies are monoclonal, preferably human or humanized, antibodies that have binding specificities for at least two different antigens or that have binding specificities for two epitopes on the same antigen. In one embodiment, one of the binding specificities is for the Wnt or Frizzled protein, the other one is for another cancer antigen. Alternatively, tetramer-type technology may create multivalent reagents.

In some embodiments, the antibody is conjugated to an effector moiety. The effector moiety can be any number of molecules, including labeling moieties such as radioactive labels or fluorescent labels, or can be a therapeutic moiety. If the effector moiety is a therapeutic moiety, it will typically be a cytotoxic agent. In this method, targeting the cytotoxic agent to cancer cells, results in direct killing of the target cell. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin, auristatin and the like. Cytotoxic agents also include radiochemicals made by conjugating radioisotopes to antibodies raised against DCAL-2 proteins, or binding of a radionuclide to a chelating agent that has been covalently attached to the antibody.

Antibodies conjugated to an effector moiety can be used both therapeutically and diagnostically, e.g., for identification of dendritic cells and monitoring of dendritic cell maturation.

### Binding Affinity of Antibodies of the Invention

Binding affinity for a target antigen is typically measured or determined by standard antibody-antigen assays, such as Biacore competitive assays, saturation assays, or immunoassays such as ELISA or RIA.

Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is <1 μM, preferably <100 nM, and most preferably <0.1 nM. Antibody molecules will typically have a K_{D} in the lower ranges. K_{D}=[Ab−Ag]/(Aberle et al., EMBO Journal, 16:3797-3804 (1997)) where (Aberle et al., EMBO Journal, 16:3797-3804 (1997)) is the concentration at equilibrium of the antibody, (Aberle et al., EMBO Journal, 16:3797-3804 (1997)) is the concentration at equilibrium of the antigen and [Ab−Ag] is the concentration at equilibrium of the antibody-antigen complex. Typically, the binding interactions between antigen and antibody include reversible noncovalent associations such as electrostatic attraction, Van der Waals forces and hydrogen bonds.

The antibodies of the invention specifically bind to DCAL-2 proteins. By “specifically bind” herein is meant that the antibodies bind to the protein with a K_{D} of at least about 0.1 mM, more usually at least about 1 μM, preferably at least about 0.1 μM or better, and most preferably, 0.01 μM or better.

### Evaluation of Dendritic Cell Function

The invention provides DCAL-2-associated modulators of dendritic cell functions that alter DCAL-2 mediated signaling. Such modulators typically bind to DCAL-2 and/or prevent binding to DCAL-2. The effects of the modulators on dendritic can be a direct phenotypic effect, e.g., on cytokine expression patterns, or expression of cell surface markers. Dendritic cell functional effects also include effects on the ability of dendritic cells to generate signals or respond to signals, e.g., signals to T-cells and B cells and/or signals generated by a pathogenic antigen.

Dendritic cell function can be assessed using many methods known in the art. Typically, in the methods of the inventions, DCAL-2-mediated dendritic cell function is assessed by determining the ability of an agent that specifically binds to DCAL-2, e.g., an antibody, to: alter DC chemokine or cytokine expression, alter the expression of cell surface or cytosolic molecules expressed on DC s, alter the ability of DCs to initiate a T-cell or B-cell response, and/or alter dendritic cell response to a pathogen and/or a product of a pathogen.

Cytokines or chemokines that can be measured include IL-1β, IL-2, IL-4, IL-6, IL-8, IL-10, IL-12p40, IL-12p70, TNF-α, MIP-3α, MIP-3β. Expression levels can be used using assays well known in the art by detecting increased in mRNA or protein expression, For example for nucleic acids, amplification reactions such as PCR, blot hybridization assays, including array analyses are commonly employed (see, e.g., Sambrook & Russell, Ausubel, both supra). For protein expression, antibody-based assays, such as ELISA or flow cytometry can be used. Of course, expression levels may also be assessed by determining the amount of activity of a protein.

Cell surface markers and certain cytosolic markers can also be determined to assess DC functions, e.g., by determining maturation, e.g., under conditions as described in the Examples section. Cell surface markers and certain cytosolic markers are commonly measured using specific monoclonal antibodies and flow cytometry. Examples of cell surface markers on dendritic cells include CD1a, CD40, CD54, CD80, CD83, CD86, CCR7, CXCR4, MHC class II, and CD209 (DC-SIGN). An example of a cytosolic marker of mature DCs detectable by flow cytometry is DC-LAMP.

The ability of dendritic cells to activate T cells and/or initiate a T-cell proliferative response can also be determined using assays known to those in the art. Exemplary assays are provided in the Examples section. Such assays include measuring T cell proliferation induced by dendritic cells in the presence of an agent that specifically binds to DCAL-2.

T cell proliferation is commonly determined using two methods. 1) [3H] thymidine incorporation and 2) labeled T cells with 5-(6)-carboxy-fluorescein succinimidyl ester (CFSE) followed by flow cytometry. Exemplary assays are performed as briefly outlined in the following passages.

Immature DCs are stimulated with graded doses of LPS, zymosan or irradiated CD40L transfected L cells in the presence or absence of anti-DCAL-2 mAb or IgM control for 24 hours and then washed. For T cell proliferation assays, CD45RA⁺ and CD45RO⁺ T cells are re separated using anti-CD45RO magnetic beads and labeled with CFSE. Labeled T cells (e.g., 98% purity) are then cultured with the pretreated DCs as and anti-CD3 mAb for 3-5 days and then analyzed by flow cytometry. In other experiments, naïve CD4+ CD45RA⁺ T cells are purified by negative selection using anti-CD8 and anti-CD45RO magnetic beads. The naïve T cells are e cultured with the pretreated DCs for 5 days then pulsed with [3H]thymidine for 18 hours to monitor T cell proliferation.

Similarly, dendritic cell-mediated T-cell maturation can also be examined. This is commonly performed by determining the presence of T-cell surface or cytosolic markers that reflect maturation measure cytokines such as IFN-γ, IL-10, IL-4 or IL-5 production or surface markers such as CD25 or CD154. In an exemplary assay to monitor T cell differentiation, anti-CD3 mAb is added to DC-T cell cultures in order to enhance T cell activation. After several days, cells are harvested and stimulated with PMA and ionomycin for 5 hours and intracellular cytokine staining or ELISA for IFN-γ and IL-4 used to detect cytokine-producing T cells, or cytokine levels.

Dendritic cell function can also be determined by assessing the ability of an agent that binds to DCAL-2 to modulate dendritic cell maturation in response to pathogens and/or products of pathogens. An for example, immature DCs are cultured in medium alone or with graded doses of E coli LPS or yeast zymosan or poly I:C along with an isotype control protein or anti-DCAL-2 mAb, then 24-48 hours later, levels of DC maturation markers such as CD83, CD86, MHC class II, DC-LAMP and CCR7 are measured, typically using flow cytometry.

B-cell function can also be measured as an assessment of dendritic cell function, as DCs can affect B cells as well as T cells (see, e.g., Carxton et al., Blood 101:4464-4471, 2003; Jego et al. Curr. Dir. Autoimmun. 8:124-139, 2005, and Banchereau et al., Immunity 20:539-550, 2004). Assays are known in the art, see, e.g., the cited references).

In order to assess the effects of a candidate agent on DCAL-2-associated DC function, assays assessing DCAL-2 function are performed in the presence of the candidate modulator, e.g., an antibody that specifically binds to DCAL-2, or another agents, such as soluble DCAL-2 receptor. Samples are compared to appropriate control samples without the agent to examine the extent of activity. Positive control samples, e.g., such as known DCAL-2 modulators, can also be included. “Activation” in the presence of a candidate modulator is achieved when the activity value relative to the control (untreated with candidate agent) is 110%, more preferably 150%, more preferably 200-500% (i.e., two to five fold higher relative to the control), more preferably 1000-3000% higher. For determining inhibitory activity, control samples (untreated with candidate agent) are assigned a relative activity value of 100%. Inhibition of a functional effect is achieved when the activity value relative to the control is about 80%, preferably 50%, more preferably 25-0%.

### Methods of Screening for Modulators of DCAL-2 Activity

The invention also provides methods of screening for modulators of DCAL-2 activity. Modulators of DCAL-2 activity are tested using DCAL-2 polypeptides, e.g., a polypeptide comprising the extracellular domain of the amino acid sequence set forth in SEq ID NO:1, either recombinant or naturally occurring. The protein can be isolated, expressed in a cell, e.g., a dendritic cell or a cell line, e.g., Chinese hamster ovary (CHO) cells, expressed in a membrane derived from a cell, expressed in tissue or in an animal, either recombinant or naturally occurring. Modulation is tested using one of the in vitro or in vivo assays described herein to analyze DCAL-2-mediated dendritic cell signaling eventss. DCAL-2 activity can also be examined in vitro with soluble or solid state reactions, using a chimeric molecule such as an extracellular domain of DCAL-2 covalently linked to a heterologous protein, or a heterologous extracellular domain covalently linked to the transmembrane and or cytoplasmic domain of DCAL-2.

The ability of a candidate compound to bind to a DCAL-2 polypeptide, a domain, e.g., the extracellular domain, or chimeric protein can be tested in a number of formats. For example, binding can be performed in solution, in a bilayer membrane, attached to a solid phase, in a lipid monolayer, or in vesicles. Often, competitive assay that measure the ability of a compound to compete with binding of the natural ligand to the receptor are used. Binding may be measured by assessing DCAL-2 activity or by other assays: binding can be tested by measuring e.g., changes in spectroscopic characteristics (e.g., fluorescence, absorbance, refractive index), hydrodynamic (e.g., shape) changes, or changes in chromatographic or solubility properties.

Candidate modulators of DCAL-2 function in dendritic cells can be any small chemical compound, or a biological entity, such as a polypeptide, sugar, nucleic acid or lipid. Typically, test compounds are small chemical molecules, peptides, including antibodies (such as monoclonal, humanized or other types of binding proteins that are known in the art), and siRNAs. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, the agents have a molecular weight of less than 1,500 daltons, and in some cases less than 1,000, 800, 600, 500, or 400 daltons. For example, agents less likely to be successful as drugs based on permeability and solubility were described by Lipinski et al. as follows: having more than 5H-bond donors (expressed as the sum of OHs and NHs); having a molecular weight over 500; having a LogP over 5 (or MLogP over 4.15); and/or having more than 10H-bond acceptors (expressed as the sum of Ns and Os). See, e.g., Lipinski et al. Adv Drug Delivery Res 23:3-25 (1997). Compound classes that are substrates for biological transporters are typically exceptions to the rule.

In some embodiments, nucleic acids such as DCAL-2 siRNA can be screened for the ability to block DCAL-2-mediated signaling events when administered in vitro or in vivo.

In one embodiment, high throughput screening methods are used. Such methods involve providing a combinatorial chemical or peptide library containing a large number of potential therapeutic compounds (potential modulator or ligand compounds). “Combinatorial chemical libraries” or “ligand libraries” are then screened in one or more assays, as described herein, to identify those library members (particular chemical species or subclasses) that display a desired characteristic activity, e.g., binding to DCAL-2. The compounds thus identified can serve as conventional “lead compounds” or can themselves be used as potential or actual therapeutics.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)). Other libraries that can be screened include nucleic acid libraries (see Ausubel, Berger and Sambrook, all supra), peptide nucleic acid libraries (see, e.g., U.S. Pat. No. 5,539,083).

### Validation of Candidate DCAL-2 Agents

Once a potential DCAL-2 modulator has been selected, typically by assessing binding activity and functional effects on DCAL-2-associated DC function, the modulator can be further validated in animal models. For example, a candidate immune enhancing agent, e.g., that binds DCAL-2 and increases DC cytokine expression such as IL-10, can be tested in vivo for the ability to enhance an immune response. This can be performed, for example, by measuring T-cell proliferative responses to a cancer or pathogenic antigen, or determining changes in T-cell maturation in the animal subject.

In some embodiments, e.g., indications where the antibody is used for the treatment of autoimmune disease, particular animal models can be used to further validate the selected candidate modulator. For example, the candidate agents can be tested in models of arthritis, either spontaneous or experimental, that are known in the art. These include collagen antibody-induced arthritis, e.g., in mice (see, e.g., McCoy et al., J Clin Invest: 110:651-658, 2002) and other well known models, such as adjuvant-induced arthritis rat models, collagen-induced arthritis rat and mouse models and antigen-induced arthritis rat, rabbit and hamster models (e.g., all described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993). The ability of the compounds to reduce the symptoms of arthritis, e.g., the time of onset, severity, etc. can be evaluated in the model by measuring parameters, well known in the art. These parameters include paw edema, histopathological evaluation of tissues, e.g., joints, measurement of levels of enzymes, such as proteases, etc.

Similarly, other art known models, e.g., rodent models of diabetes, can be used to validate candidate DCAL-2 modulators for the treatment of diabetes. Further, animal models of cancer and infectious disease models are used to evaluate the effects of a DCAL-2 reagent, e.g., antibody, on the ability to enhance immune responses to cancer and pathogenic antigens.

### Administration of DCAL-2 Modulators to Modulate Immune Response

Agents that modulate DCAL-2-related dendritic cell function (e.g., antibodies) can be administered by a variety of methods including, but not limited to parenteral (e.g., intravenous, intramuscular, intradermal, intraperitoneal, and subcutaneous routes), topical, oral, local, aerosol, or transdermal administration.

Various delivery systems are known and can be used to administer DCAL-2 signaling modulators of the invention, e.g., encapsulation in liposomes, microparticles, microcapsules, recombinant cells capable of expressing antibody or peptide modulators, receptor-mediated endocytosis (see, e.g., Wu and Wu, J. Biol. Chem. 262:4429-4432, 1987), construction of a nucleic acid as part of an adenoviral or other vector, etc. The compositions may be administered by any convenient route, for example by infusion or bolus injection, by absorption through epithelial or mucocutaneous linings (e.g., oral mucosa, rectal and intestinal mucosa, etc.), and may be administered together with other biologically active agents. Administration can be systemic or local.

In one embodiment, the DCAL-2 signaling modulator can be delivered in a controlled release system. In one embodiment, a pump may be used. In another embodiment, polymeric materials can be used In specific embodiments, a controlled release system can be placed in proximity of the therapeutic target, e.g., an affected organ of the body, such as the brain, lungs, kidney, liver, ovary, testes, colon, pancreas, breast, and skin, thus requiring only a fraction of the systemic dose (see, e.g., Goodson, in Medical Applications of Controlled Release, vol. 2, pp. 115-138. Controlled release systems are widely known in the art (see, e.g., Langer, Science 249:1527-1533, 1990; Sefton, CRC Crit. Ref. Biomed. Eng. 14:201, 1987; Buchwald et al., Surgery 88:507, 1980; Saudek et al., N. Engl. J. Med. 321:574, 1989; Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J. Macromol. Sci. Rev. Macromol. Chem. 23:61, 1983; Levy et al., Science 228:190, 1985; During et al., Ann. Neurol. 25:351, 1989); and Howard et al., J. Neurosurg. 71:105, 1989).

DCAL-2 modulators can be used to modulate the immune response in any number of disease, including infectious disease, cancer, and autoimmune disease. The compositions for administration will commonly comprise a modulator provided in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs.

The amount of the modulator of DCAL-2 signaling that will be effective in the treatment, inhibition, lessening, or prevention of a disease or disorder associated with activity of dendritic cells can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems. Actual methods for preparing compositions for administration in accordance with the invention will be known or apparent to those skilled in the art and are described in more detail in such publications as Remington's Pharmaceutical Science, 15th ed., Mack Publishing Company, Easton, Pa. (1980).

Toxicity and therapeutic efficacy of DCAL-2 signaling modifying compounds can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a modulator is from about 1 ng/kg to 10 mg/kg for a typical subject.

Often, e.g., for antibodies or peptides, dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, human antibodies can be administered in smaller dosages and with less frequent administration. Further, the dosage and frequency of administration antibodies of the invention may be reduced by enhancing uptake and tissue penetration of the antibodies by modifications such as, for example, lipidation.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that antibodies or other polypeptides when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions containing modulators of the invention (e.g., antibodies or peptides) can be administered for therapeutic or prophylactic treatments. In therapeutic applications, compositions are administered to a patient suffering from a disease (e.g., cancer) in an amount sufficient to cure or at least partially arrest or delay the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient.

An amount of an inhibitor that is capable of preventing or slowing the development of a disease in a patient is referred to as a “prophylactically effective dose.” The particular dose required for a prophylactic treatment will depend upon the medical condition and history of the patient, the particular cancer being prevented, as well as other factors such as age, weight, gender, administration route, efficiency, etc. Such prophylactic treatments may be used, e.g., in a patient who has previously had cancer to prevent a recurrence of the cancer, or in a patient who is suspected of having a significant likelihood of developing cancer, or a patient that is at risk for infection with a pathogenic organism.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

In some embodiments, nucleic acids such as siRNAs, antisense, ribozymes and the like can be administered to modulate DCAL-2 signaling events. For example, small interfering RNAs to DCAL-2 can be administered to block DCAL-2-mediated effects. In mammalian cells, introduction of long dsRNA (>30 nt) often initiates a potent antiviral response, exemplified by nonspecific inhibition of protein synthesis and RNA degradation. The phenomenon of RNA interference is described and discussed, e.g., in Bass, Nature 411:428-29 (2001); Elbahir et al., Nature 411:494-98 (2001); and Fire et al., Nature 391:806-11 (1998), where methods of making interfering RNA also are discussed. The siRNAs based upon the DCAL-2 sequences disclosed herein are less than 100 base pairs, typically 30 bps or shorter, and are made by approaches known in the art. Exemplary siRNAs according to the invention could have up to 29 bps, 25 bps, 22 bps, 21 bps, 20 bps, 15 bps, 10 bps, 5 bps or any integer thereabout or therebetween. Such siRNAs can be administered, e.g., in a form encoded by a vector or as a liposome nucleic acid complex. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugqte Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Modulation of Immune Responses

In preferred embodiments, the DCAL-2 modulators are used in conjunction with other agents, e.g, vaccines, to modulate the immune response. Vaccines can include—cancer vaccines or vaccines to immunize against infectious agents, including viruses, bacteria, yeast or any other pathogen. Such vaccines can be administered as polypeptides or as polynucleotide-based vaccines. Regardless of the specific features of a given vaccine, they all have in common the capacity to stimulate an immune response to the antigens either encoded by the nucleic acid in a nucleic acid vaccine, or present as polypeptide sequences. The antigenic portion(s) of the vaccine may be delivered in the form of peptides, proteins, and fusion proteins, as disclosed herein above, and/or may be delivered in the form of a polynucleotide such as, for example, an RNA, a DNA and/or a virus such as adenovirus, adeno-associated virus, vaccinia virus or any other virus known in the art.

The vaccines can be directed against cancer antigens, including cancer antigens associated with breast cancer, prostate cancer, lung cancer, colorectal cancer, cervical cancer, ovarian cancer, pancreatic cancer, gastric cancer, esophageal cancer, head and neck cancer, hepatocellular carcinoma, melanoma, glioma, glioblastoma, or lymphoma. Such cancer antigens are known in the art.

The DCAL-2 reagents can also be administered with vaccines to various pathogens. Such pathogens include viruses, e.g., hepatitis B, hepatitis C, herpes, HIV; bacterial pathogens, fungi, parasites, and other pathogens where it is desired to enhance the immune response to antigens on the pathogens.

In some embodiments, a modulator that effects DCAL-2, e.g., an antibody that specifically binds to DCAL-2, can be used to treat dendritic cells ex vivo. The dendritic cells can then be returned to a patient to enhance an immune response. The ex vivo treatment can also employ exposing the dendritic cells to an antigen, e.g., using a nucleic-acid based vaccine where a polynucleotide encodes the antigen of interest, or pulsing the cells with antigenic peptides. Thus, DCAL-2 reagents can be used to modulate immune responses using ex vivo methodologies as well as in vivo methodologies.

In employing DCAL-2 reagents, e.g., antibodies, as noted above, the antibodies can be administered with or without a vaccine. In particular embodiments of the invention, the methods comprises administering an antibody, e.g., a humanized monoclonal antibody, to a patient that has cancer, with the proviso that the cancer is not leukemia, e.g., acute myelogenous leukemia, or lymphoma. For example, such a cancer can be a cancer that involves solid tumors, such as adenocarcinomas, sarcomas, and the like, including cacncer such as breast, prostate, colorectal, lung, pancreatic, ovarian, cervical, esophageal, gastric, hepatic, and various sarcomas.

In some embodiments, DCAL-2 signaling modulators, e.g., antibodies, can be administered for the treatment of autoimmune diseases. In the context of autoimmune disease, a DCAL-2 signaling modulator of the invention can be administered for the treatment of the disease based on such criteria as the expression pattern of cytokines (see, e.g., the review by Banchereau et al., Immunity 20, 539-550, 2004). Thus, an appropriate modulator can be selected based on the “type” of autoimmune disease. For example, DCAL-antibodies can be administered to patients with particular autoimmune diseases that have been characterized as “TH-1-like” diseases. These include arthritis, multiple sclerosis, psoraisis, rheumatoid arthritis, insulin-dependent diabetes.

The modulators may also be used in some contexts, for treatment of “TH-2-like” diseases, such as asthma and autoimmune hemolytic anemia. For example, the blockade of a DCAL-2 pathway by soluble DCAL-2 may promote a TH1-like response and reduce destructive TH2-like responses present in asthma and atopic diseases.

The modulators of DCAL-2 signaling can also be administered to patient with chronic inflammatory disease. Thus, autoimmunte disorders that can be treated with the modulators of the invention include arthritis, e.g., rheumatoid arthritis, osteoarthritis, juvenile chronic arthritis, and other inflammatory diseases of the joint; inflammatory bowel diseases, e.g., ulcerative colitis, Crohn's disease, Barrett's syndrome, ileitis, enteritis, and gluten-sensitive enteropathy; vaginitis; inflammatory disorders of the respiratory system, such as asthma, adult respiratory distress syndrome, allergic rhinitis, chronic obstructive airways disease, hypersensitivity lung diseases and the like; inflammatory diseases of the skin, including psoriasis, scleroderma, and inflammatory dermatoses such as eczema, atopic dermatitis, urticaria, and pruritis; disorders involving inflammation of the central and peripheral nervous system, including multiple sclerosis, idiopathic demyelinating polyneuropathy, Guillain-Barre syndrome, chronic inflammatory demyelinating polyneuropathy, neurodegenerative diseases such as Alzheimer's disease.

Various other inflammatory diseases or immune-related diseases can also be treated using the methods of the invention. These include autoimmune diseases such as systemic lupus erythematosis, immune-mediated renal disease, e.g., glomerulonephritis, diabetes mellitus, and spondyloarthropathies; and diseases with an undesirable inflammatory component such as systemic sclerosis, idiopathic inflammatory myopathies, Sjogren's syndrome, vasculitis, sarcoidosis, thyroiditis, gout, otitis, conjunctivitis, sinusitis, sarcoidosis, Behcet's syndrome, hepatobiliary diseases such as hepatitis, primary biliary cirrhosis, granulomatous hepatitis, and sclerosing cholangitis; inflammation and ischemic injury to the cardiovascular system such as ischemic heart disease, stroke, and atherosclerosis; and graft rejection, including allograft rejection and graft-v-host disease. Various other inflammatory diseases are described, e.g., in Harrison's Principles of Internal Medicine, 12th Edition, Wilson, et al., eds., McGraw-Hill, Inc.).

Modulators, e.g., blocking DCAL-2 blocking agents can also be used to treat allergic reactions and conditions (e.g., systemic anaphylaxis or hypersensitivity responses, drug allergies, food allergies, insect sting allergies, allergic contact dermatitis, etc.).

### Modulating an Immune Response

The ability of DCAL-2 reagents to modulate an immune response, either in conjunction with a vaccine or when administered separately, can be evaluated using known techniques (see, e.g., the Examples section for exemplary assays). Such techniques typically measure T-cell proliferation, T-cell differentiation markers, or activation of T-cells, e.g., cytotoxic T cells, or B cell activities. Such assays can be used, e.g., to monitor a subject's response to the DCAL-2 reagent (with or without other therapeutic agents.

Modulation of an immune response by a DCAL-2 reagent, e.g., administration of a DCAL-2 antibody, is typically characterized by changes in at least one of several measurable endpoints. The changes can reflect stimulation of an immune response, or can also be used, as appropriate, to measures inhibition of an immune response, e.g., for the treatment of autoimmune disorders. These changes reflect dendritic cell function, and thus includes, e.g., measuring changes in maturation of DCs in the presence of a pathogen or antigen from a pathogen; assessing alterations in the ability of isolated DCs from a treated subject to induce T cell proliferative responses in comparison to controls; measuring changes in the ability to mount a T cell proliferative response against a subsequent challenge with antigen; a changed immune responses with respect to either TH2 or Th1 immunity to pathogens (see, e.g., Pulendran et al. Science 293:253-256, 2001); and the like. Thus, the effects of administering a DCAL-2 modulators, e.g., an antibody or DCAL-2 soluble receptor, can be readily determined.

### EXAMPLES

Materials and Methods

### Primary Cell Culture and Cell Lines

CD14⁺ monocytes were isolated from leukopheresis products of healthy donors (Fred Hutchinson Cancer Research Center, WA) by positive magnetic selection as described by the manufacturer (Miltenyi Biotech, Auburn, Calif.). These CD14⁺ cells were then differentiated in culture for 5-7 days into monocyte-derived immature DCs (iDCs) using IL-4 (30 ng/ml) (Research Diagnostics Inc., Flanders, N.J.) and GM-CSF (100 ng/ml) (Amgen, Seattle, Wash.). To induce maturation, iDCs were stimulated with various doses of E. coli LPS (Sigma-Aldrich Corp, St Louis, Mo.), Yeast Zymosan (Sigma-Aldrich Corp, St Louis, Mo.), CD40L transfected L cells for 24 h or 48 h. The phenotype of the DCs before and after stimulation was established by flow cytometry using a FACScan analyzer (Becton Dickinson). Immature DC were defined as CD14⁻ CD1a^{high} CD80⁺ CD86⁺ and mature DCs were defined as CD14⁻ CD1a^{high} CD80^{high} CD86^{high} CD83⁺ HLA-DR⁺⁺. CD1a⁺ and BDCA-2⁺ cells were purified from PBMC using anti-CD1a and anti-BDCA-2 magnetic beads (Miltenyi, Auburn, Calif.). Dense human tonsillar B cells and peripheral blood B cells and CD3⁺ T cells obtained from peripheral blood mononuclear cells (PBMCs) were prepared as previously described (Clark, et al., J Immunol 143:3873, 1989; Valentine, et al., J Immunol 140:4071, 1988). T cells were activated for 24 hours with a mAb to CD3 (64.1) at 1 μg/ml in solution. B cells were activated with 1 μg/ml anti-CD40 (G28-5) and 10 μg/ml anti-IgM (Jackson Immunoresearch Laboratories Inc. West Grove, Pa.) for 24 hours. The BJAB, Daudi, HL-60, Jurkat, MP-1, Nalm-6 and U937 cell lines were cultured in RPMII 640 supplemented with 10% FCS at 37° C. and 5% CO₂.

Northern Blot Analysis of hDCAL-2

Commercially available membranes containing 2 μg of mRNA were purchased from Clontech and used as described by the manufacturer. Briefly, the blot was hybridized with a ³²P-labeled full length hDCAL-2 cDNA probe and washed twice for 20 minutes with 2×SSC/0.05% SDS at room temperature and then with 0.1% SSC/0.1% SDS at 58° C. The membranes were subsequently stripped and re-probed using radioactively labeled human β-actin cDNA probe (Clontech).

RT-PCR Expression Analysis of hDCAL-2

To prepare RNA from the purified primary cells or cell lines, cells were either lysed in Trizol (Invitrogen) and RNA was isolated as described by the manufacturer or directly isolated by using Qiagen Rneasy kit. First strand cDNA synthesis was performed using oligo dTs and AMV reverse transcriptase (Promega, Madison, Wis.) in standard reverse transcription reactions. Human DCAL-2 expression was analyzed by PCR of the cDNA using the following specific primers: 5′cattcagctctgttaactcactcatctt-3′ (SEQ ID NO:6) and 3′aggcagaggagttgattatattatccac-5′ (SEQ ID NO:7) and 30 cycles of 94° C. for 30 seconds, 50° C. for 30 seconds, 72° C. for 45 seconds. β-Actin primers were used as loading controls, forward 5′gtcgtcgacaacggctccggcatctg3′ (SEQ ID NO:8) and reverse-3′cattgtagaaggtgtggtgccagatc-5′ (SEQ ID NO:9).

### Generation of DCAL-2-His Protein and Monoclonal Antibody

The extracellular region of DCAL-2 was cloned into pQE31 vector of QIAExpress kit (Qiagen, Chatsworth, Calif.) using the following primers: DCAL-2 (BamH I), 5′-GTTGTTGGATCCGGCAAGCATGTTTCATGT-3-[[″]]′ (SEQ ID NO:10) and (HindIII) 5′-CGCAAGCTTTGTTGCCTCCCTAAAATATGTA-3′ (SEQ ID NO:11) to make the expression construct for the six-His-tagged DCAL-2. E. coli M15 (pREP4) was used to express the fusion protein in Luria-Bertonia broth supplemented with ampicillin (100 mg/ml), Kanamycin (25 mg/ml) and 1 mM isopropyl-β-D-galactopyranoside (IPTG). The protein then was solubilized in urea and purified using a nickel-nitrilo-triacetic acid resin column (Qiagen) according to the manufacture.

To generate monoclonal antibodies (mAbs) against human DCAL-2, BALB/c mice were immunized with DCAL-2-His formulated with monophosphoryl lipid A and trehalose dicorynomycolate emulsion (Corixa, Hamilton, Mont.) as adjuvant. Mice were boosted twice at weeks 2 and 10. Three days following the final boost, spleens were removed and fused with NS-1 cells to make hybridomas. Positive clones were determined by ELISA screen using the DCAL-2 fusion protein and FACS analysis using CD14⁺ cells. Isotypes of monoclonal antibodies were determined by using Cytometric Bead Array (CBA) kit from BD PharMingen (San Diego, Calif.) according to the manual. One IgM monoclonal antibody, UW70, was established and found to be specific for DCAL-2.

### Cell Surface Expression of DCAL-2

Various cell lines were incubated with anti-DCAL-2 (UW70) or IgM isotype control, followed by FITC conjugated goat F(ab′)₂ anti-mouse IgM+IgG (Jackson Immunoresearch Laboratories Inc. West Grove, Pa.). To investigate the expression of DCAL-2 on primary cells, PBMCs from health donors were first labeled with anti-DCAL-2 mAb, washed, and then stained with FITC conjugated goat F(ab′)₂ anti-mouse (IgM+IgG) or PE-conjugated anti-mouse IgM, then washed and incubated with PE- and FITC- conjugated anti-CD3, anti-CD20, anti-CD16 or anti-CD14 mAb. To stain DCAL-2 on blood DC and pDC, CD1a⁺ and BDCA-2⁺ cells were purified from PBMC using anti-CD1a and anti-BDCA-2 magnetic beads.

### Internalization Assay

Approximately 1 million iDCs were incubated with anti-DCAL-2 or control IgM for 30 min at 4° C. After washing twice with cold PBS, cells were re-suspended and cultured with warm media (37° C.). At the indicated time points, cells were washed and then fixed by adding 4% of paraformaldehyde. The level of cell surface-remained anti-DCAL-2 were detected using FITC-conjugated goat F(ab′)₂ anti-mouse (IgM+IgG). To study if ligation of DCAL-2 would affect endocytosis, iDCs were pretreated with anti-DCAL-2 for 30 min at 4° C., and then incubated with FITC-conjugated Dextran at 37° C. Cells were fixed at the indicated time points and the levels of FITC-dextran were analyzed using a FACScan.

### Western Blotting

For anti-phosphotyrosine blots, iDCs were incubated with anti-DCAL-2 mAb for 30 min at 4° C. After washing twice with cold PBS, cells were re-suspended with warm medium (37° C.) and lysed at the indicated time points by lysis buffer. For MAPK activation, iDCs were stimulated with LPS (1 μg/ml) or Zymosan (100 μg/ml) in the presence or absence of anti-DCAL-2 mAb for the indicated time points at 37° C. then the cells were lysed. Total protein concentrations were measure using a Bio-Rad protein kit (Bio-Rad, Inc, Hercules, Calif.). Equal amounts of protein were mixed with the Laemmli protein sample buffer and boiled, then separated by SDS-PAGE and transferred to nitrocellular membranes. After blocking with 5% fat-free milk, the blots were probed with antibodies against phosphotyrosine (4G10), anti-phospho-p38 MAPK, anti-phospho-ERK, anti-phospho-JNK, anti-phospho-IκB or anti-phospho-SHP. The blots were washed and incubated with horseradish peroxidase (HRP)-labeled secondary Abs. Blots were then visualized by an ECL detection reagent. To ensure similar protein loading, the blots were stripped and re-probed with anti-total p38 MAPK as loading control.

### Cytokine Analysis

Immature DCs were stimulated with LPS, zymosan or CD40L transfected L cells in the presence or absence of anti-DCAL-2 or IgM isotype control for 24 hours or 48 hours. The supernatants were collected, and then cytokines/chemokines were analyzed using human cytokine antibody arrays (Raybiotech, Inc. Norcross, Ga.) and ELISA (IL-6, IL-10, IL-12 p40, IL-12 p70, TNF-α and MIP-3p, R&D Systems, Inc. Minneapolis, Minn.).

### T Cell Proliferation and Differentiation

Immature DCs were stimulated with graded doses of LPS, zymosan or irradiated CD40L transfected L cells in the presence or absence of anti-DCAL-2 mAb or IgM control (10 μg/10⁶ cells) for 24 hours and then washed. For T cell proliferation assays, CD45RA⁺ and CD45RO⁺ T cells were separated using anti-CD45RO magnetic beads and labeled with 5 μM of CFSE. Labeled T cells (98% purity) were then cultured with the pretreated DCs as described above and anti-CD3 mAb (1 μg/ml) for 5 days and then analyzed by flow cytometry. In other experiments, naïve CD4⁺ CD45RA⁺ T cells were purified by negative selection using anti-CD8 and anti-CD45RO magnetic beads. The naïve T cells were cultured with the pretreated DCs for 5 days then pulsed with [³H] thymidine for 18 hours to monitor T cell proliferation. To monitor T cell differentiation, anti-CD3 mAb (1 μg/ml) was added to DC-T cell cultures in order to enhance T cell activation. After 5 days, cells were harvested and stimulated with PMA and ionomycin for 5 hours and intracellular cytokine staining or ELISA for IFN-γ and IL-4 were used to detect cytokine producing T cells or cytokine levels.

### Results

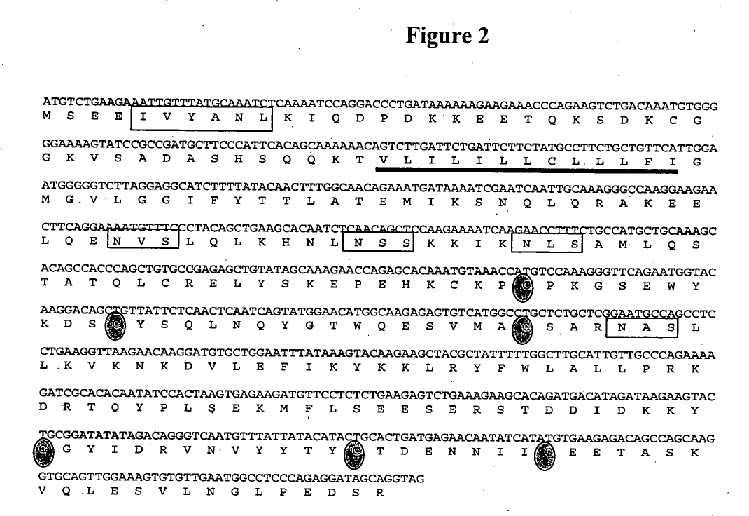

DCAL-2, a type II C-type lectin, is restricted in its expression to human monocytes and myeloid dendritic cells. Human DCAL-2 was identified by using the program blastpgp to create position-specific scoring matrices (PSSMs) based on the carbohydrate recognition domain (CRD) of DC-SIGN and DCIR then blasted against human EST databases. Comparisons between hDCAL-2 protein sequence databases and BLAST alignments revealed features typical of a type II transmembrane C-type lectin with a cytoplasmic tail that contains a tyrosine at position 7 centered in the sequence VTYADL (SEQ ID NO:12). This is identical to the consensus sequence I/VXYXXL/V of an immunoreceptor tyrosine-based inhibitory motif, ITIM. Human DCAL-2 protein also has a putative hydrophobic transmembrane region (residues 44-60) and an extracellular region containing a single carbohydrate recognition domain (CRD) with 6 cysteines to form 3 disulphide bonds. However, there is no clear calcium-binding motif within the CRD. There are however, 6 potential glycosylation sites, at amino acid positions 78, 86, 96, 103, 156 and 217 (FIG. 1A). The analysis of the genomic sequence indicates that DCAL-2 is located at human chromosome 12p13 in a region near a cluster of C-type lectins and the gene is composed of 6 exons (FIG. 1B). Mouse DCAL-2 was also identified from partial ESTs and the full-length sequence obtained from 5′ and 3′RACE PCR. When compared to the human sequence of DCAL-2, mDCAL-2 had similar genomic structure and the nucleotide sequence is 72% homologous to hDCAL-2 suggesting these are homologous molecules. FIG. 2 shows the mouse DCAL-2 sequence, including conserved regions that are identical or represent conservative modifications. Comparisons between the primary protein structure of DCAL-2 and other C-type lectins show hDCAL-2 has the greatest identity to the human lectins, ! Dectin-1 (31%) CLEC-1 (30%) and CLEC-2 (30%), with lower homology to DCIR (24%), CD69 (21%) and DCAL-1 (11%). The phylogenetic tree of these similar C-type lectins indicates hDCAL-2 forms a cluster with Dectin-1, CLEC 1 and CLEC 2, (FIG. 1C).

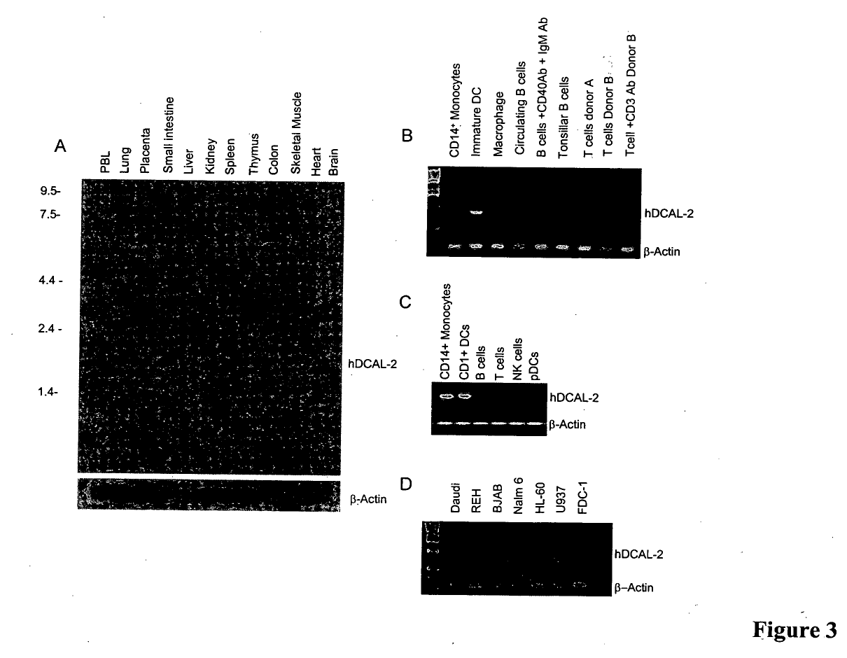

The expression of DCAL-2 in human tissues was determined by Northern blot and showed a single band at about 1.6 Kb (FIG. 3A). DCAL-2 mRNA was detected at highest levels in peripheral blood leukocytes and at lower levels in the lung, but was not detectable in placenta, liver, spleen, small intestine, kidney, colon, skeletal muscle and brain, indicating a preferential expression of hDCAL-2 in some hematopoietic tissues. RT-PCR analysis of primary cells showed hDCAL-2 is restricted in its expression to CD14⁺ monocytes, DCs and macrophages. There was no expression in NK cells, plasmacytoid DC (pDC), CD3+ T cells, activated CD3⁺ T cells stimulated with anti-CD3, peripheral blood B cells, dense tonsillar B cells or tonsillar B cells stimulated with anti-CD40 (FIGS. 3B and 3C). RT-PCR of cell lines confirmed this restricted expression pattern. Weak mRNA expression of hDCAL-2 was detected in myeloid cell lines, HL60s, U937 and a B cell line, Nalm6 (FIG. 3D). It was not detected in any other B cell lines (Daudi, REH, BJAB, CESS, MP-1, Raji, Ramos, REH, RPMI-8226) or T cell lines (CEM, Jurkat and Molt-4). It was also not detected in non-hematopoietic cells, including a stromal cell line (FDC-1), or epithelial cell lines, (HeLa) and primary epithelial cells (data not shown). These results are consistent and extend previous results of (Marshall et al., J. Biol. Chem. 279:14792-14802, 2004) that reported that human MICL RNA is predominantly expressed on myeloid cells.

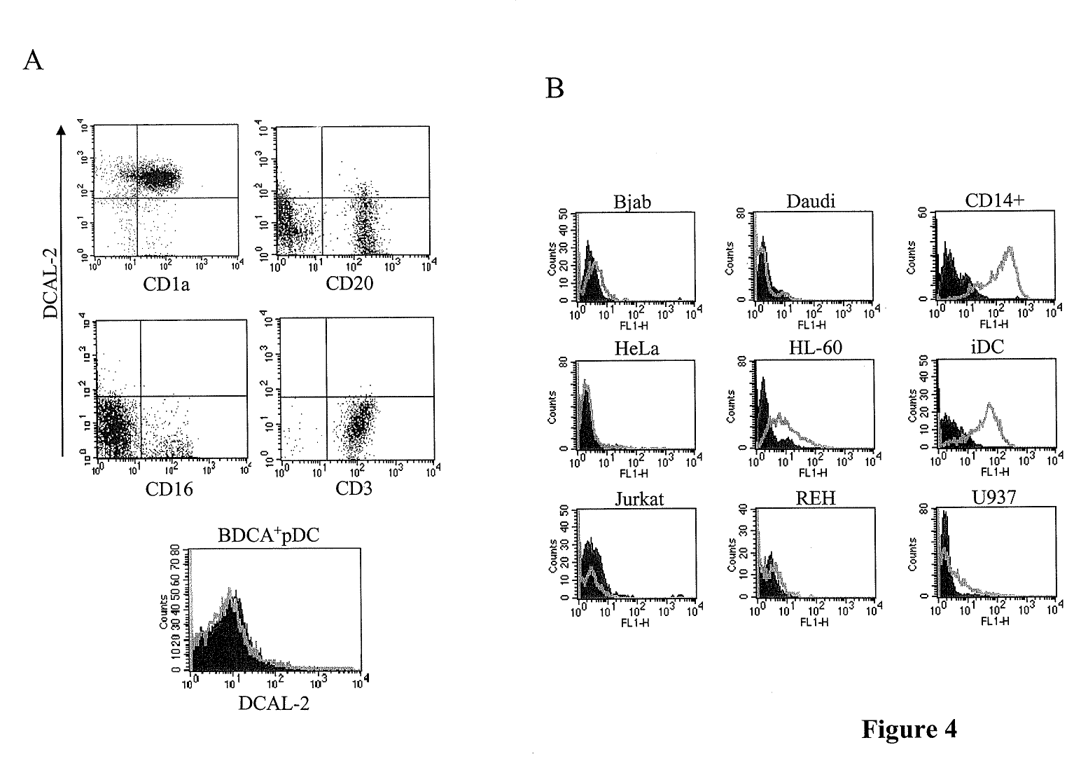

DCAL-2 protein expression was determined using the monoclonal antibody described above. Using anti-DCAL-2 mAb (IgM isotype, UW70), DCAL-2 was detected on CD14⁺ monocytes, CD1a⁺ blood DCs and monocyte-derived DCs, but not on pDCs, CD3⁺ T cells, CD16⁺ NK cells and B cells (FIG. 4A). When cell lines were tested, only myeloid cell lines such as HL60 and U937 showed weak expression of hDCAL-2; HeLa cells or B or T cell lines did not (FIG. 4B).

Tyrosine Phosphorylation is induced upon crosslinking of DCAL-2 with mAb

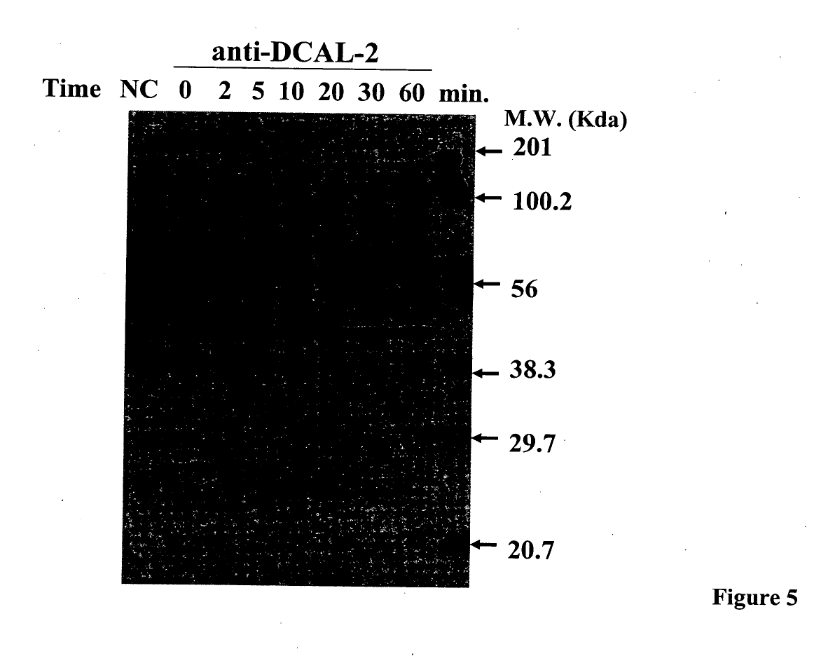

The ITIM motif on DCIR has been shown to be associated with inhibitory adaptor proteins such as SHP-1 or SHP-2 after ligand binding and to suppress B cell activation. To investigate the possible signaling function of DCAl-2, the UW70 mAb was used as a pseudo-ligand to cross-link DCAL-2 on iDCs. Total tyrosine phosphorylation at different time points was analyzed by western blotting. As shown in FIG. 5, at 5 min after DCAL-2 ligation several changes in the total tyrosine phosphorylation patterns were evident. A 100 kDa band was gradually diminished up to the 60 min time point. Several major tyrosine phosphorylated protein bands were induced after DCAL-2 binding with molecular weights of around 90, 70 and 60 kDa as well as a band at around 45 kDa. Because the UW70 mAb did not immunoprecipetate DCAL02 or identify it on a western blot DCAL-2, it could not be determined in this study whether one of these bands was DCAL-2 or one of the tyrosine phosphorylated proteins bound to DCAL-2 after ligand binding. However, these data suggest that DCAL-2 possesses a functional signaling motif and that ligand binding of DCAL-2 may signal DCs.

### DCAL-2 is Internalized After Ligand Binding

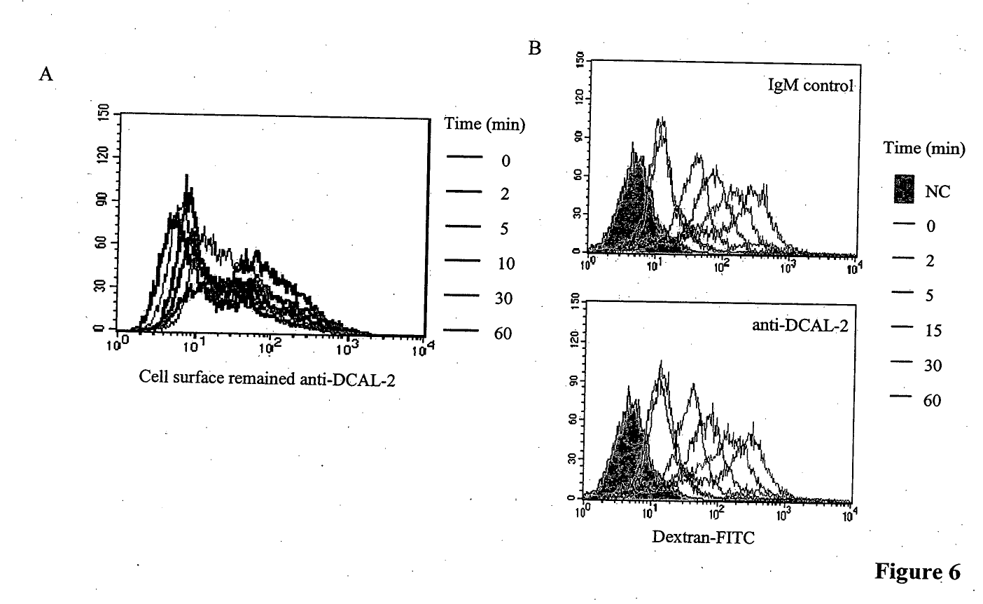

Several C-type lectins have the capacity for antigen uptake after ligand binding. although several internalization motifs involved in receptor internalization have been identified, the cytoplasmic tail of DCAL-2 does not appear to have any of the known motifs. However, it has been suggested that C-type lectins may associate with adaptor proteins that facilitate internalization or signaling. To investigate whether DCAL-2 may be involved in antigen uptake, iDC were coated with anti-DCAL-2 mAb at 4° C., washed and then warmed to 37° C. for the indicated time points to allow for possible ligand-receptor internalization. Cells were fixed after the indicated time points and the amount of anti-DCAL-2 mAb remaining on the cell surface was detected by FITC-conjugated Goat F(ab′)₂ anti-mouse (IgG+IgM) and flow cytometry. A gradual internalization of DCAL-2 was observed after mAb binding, suggesting that DCAL-2 can be internalized by iDCs after ligand binding (FIG. 6A).

One of important functions of iDC is antigen uptake via different receptors. Since cross-link of DCAL-2 could signal cells, we next tested whether DCAL-2 ligation could affect antigen uptake by iDCs. FITC-conjugated Dextran-beads can be taken up by iDCs via endocytosis and have been used to study antigen capture and cellular trafficking. Anti-DCAL-2 treated iDCs were fed FITC-dextran beads for certain times, then the level of intracellular dextran beads was measured by flow cytometry. Similar increasing levels of FITC-dextran uptake can be observed in anti-DCAL-2 treated iDCs and IgM control treated iDCs, suggesting that cross-linking of DCAL-2 did not affect endocytotic-mediated dextran uptake by iDCs (FIG. 6B).

### DCAL-2 Signaling Does Not Inhibit LPS/Zymosan-Induced DC Maturation.

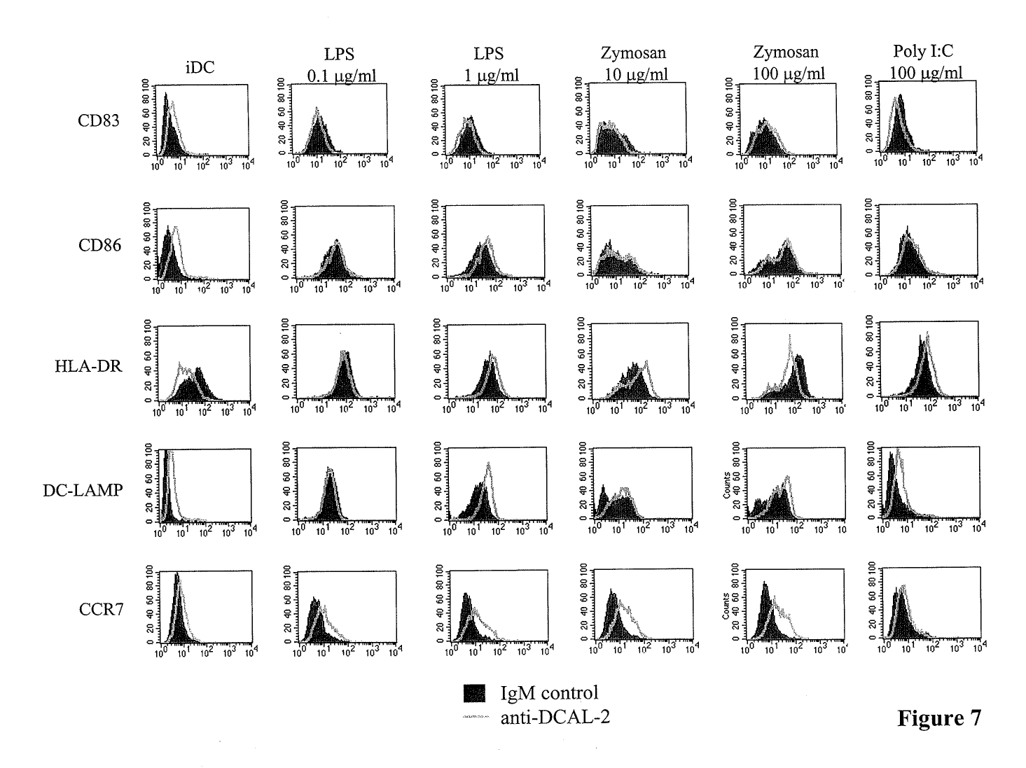

After encountering pathogens, iDCs undergo a maturation process and migrate to secondary lymphoid tissues. At this stage, the function of DCs switches from antigen capture toward antigen presentation and T cell programming. DC maturation is associated with increased expression of co-stimulatory molecules such as CD80 and CD 86, the chemokine receptor CCR7, MHC class II and DC-LAMP, an intracellular protein associated with lysosomes. To investigate whether DCAL-2 participates in regulating Toll-like receptor (TLR)-induced DC maturation, iDCs were stimulated with LPS (a stimulator of TLR4), yeast zymosan (a stimulator of TLR2) or poly I:C (a stimulator of TLR3) in the presence or absence of anti-DCAL-2 mAb for 24 hours. Maturation markers were then analyzed. Anti-DCAL-2 alone did not dramatically affect DC maturation as measured by changes in cell surface markers or DC-LAMP (FIG. 7, left). Stimulation of iDCs with either LPS or zymosan induced increased levels of CD83, CD86, HLA-DR and DC-LAMP as expected and slightly increased CCR7 expression. Polyl:C stimulation also up-regulated CD86, HLA-DR and CCR7 expression, but not DC-LAMP (FIG. 7, right). Surprisingly, the presence of anti-DCAL-2 significantly increased LPS— and zymosan-induced DC maturation as measured by CCR7 and DC-LAMP expression, but had less of an effect on polyI:C stimulated DCs. Slight increases in CD86 or HLA-DR were observed when low doses of zymosan or polyI:C were combined with anti-DCAL-2 mAb. These results suggest that DCAL-2 interacts directly or indirectly with different TLRs during DC maturation. The combination of DCAL-2 with certain TLRs such as TLR2 and TLR4 augments DC maturation process with regard to DC migration and antigen processing.

### Modulation of DC Cytokine/Chemokine Expression by DCAL-2 Signaling.

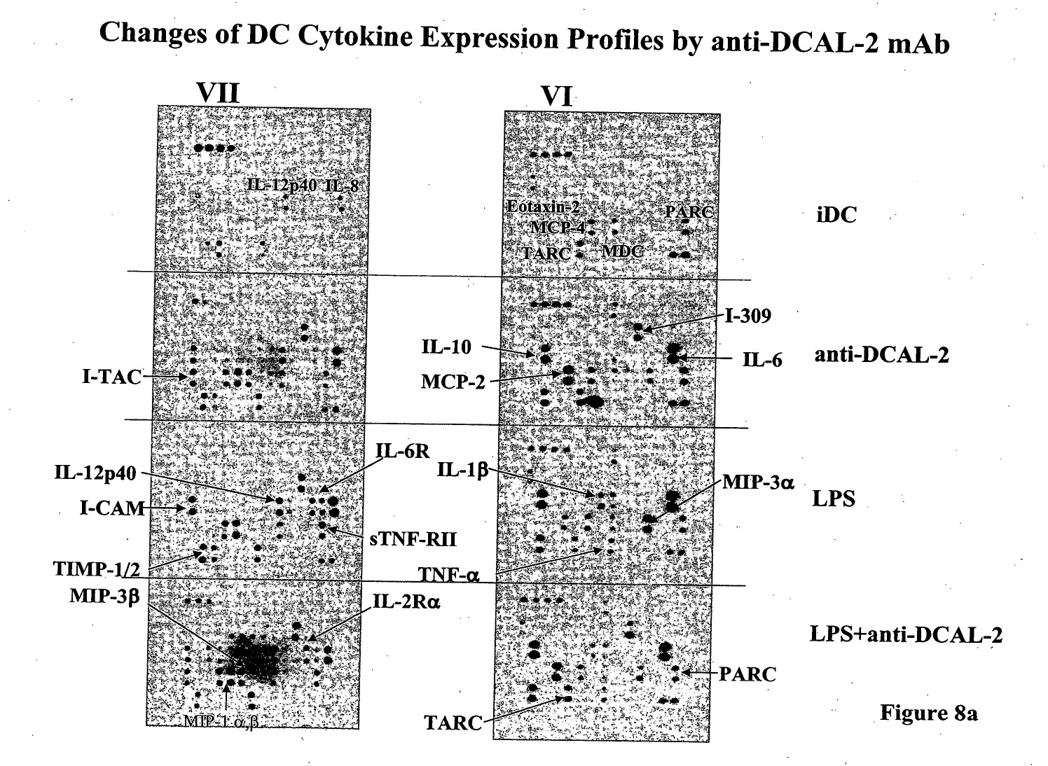

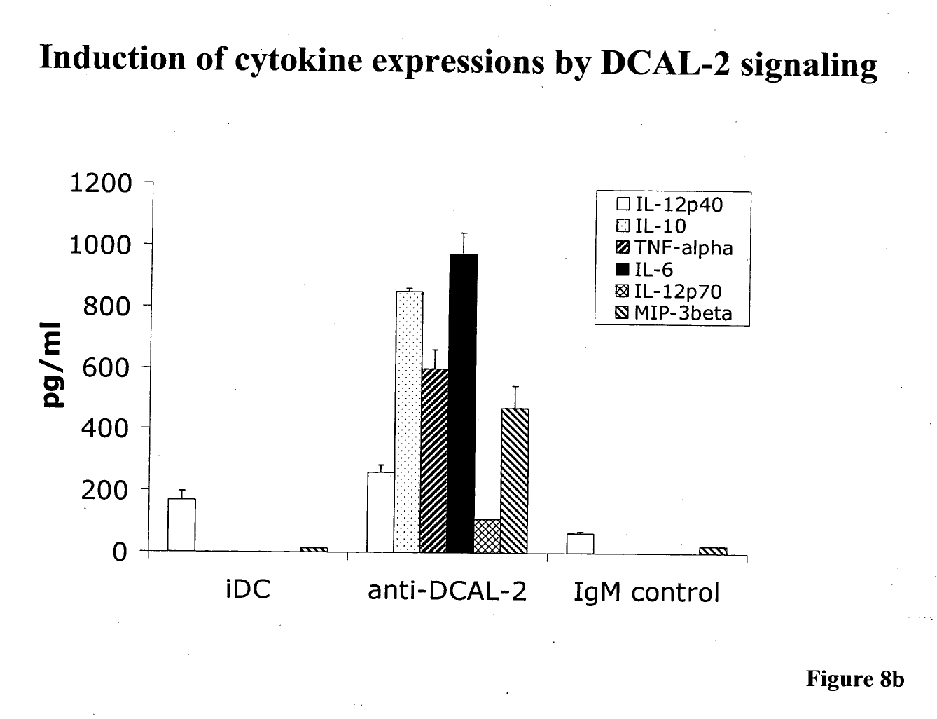

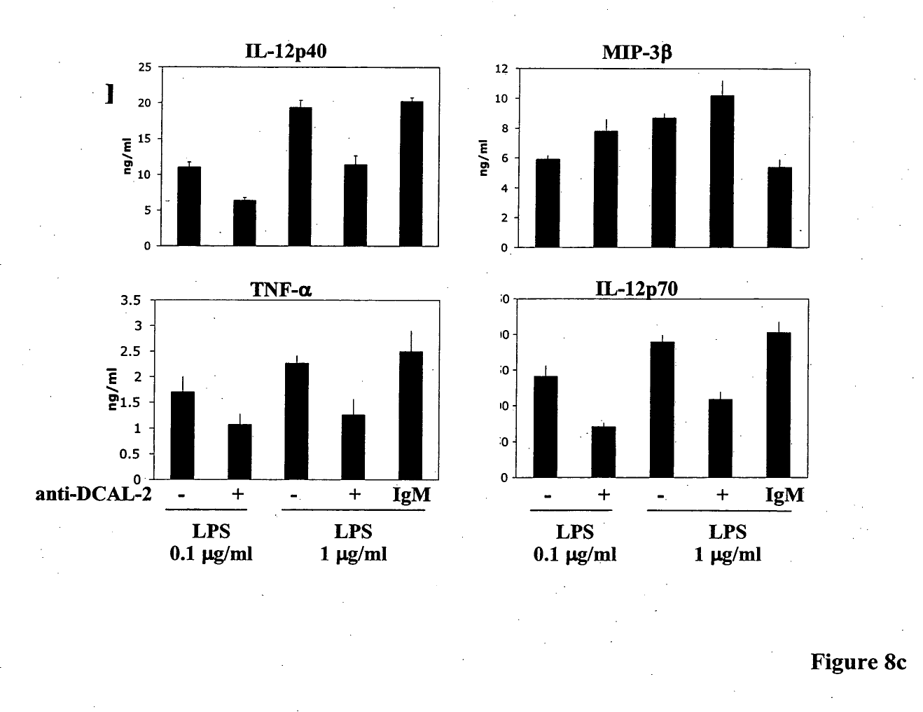

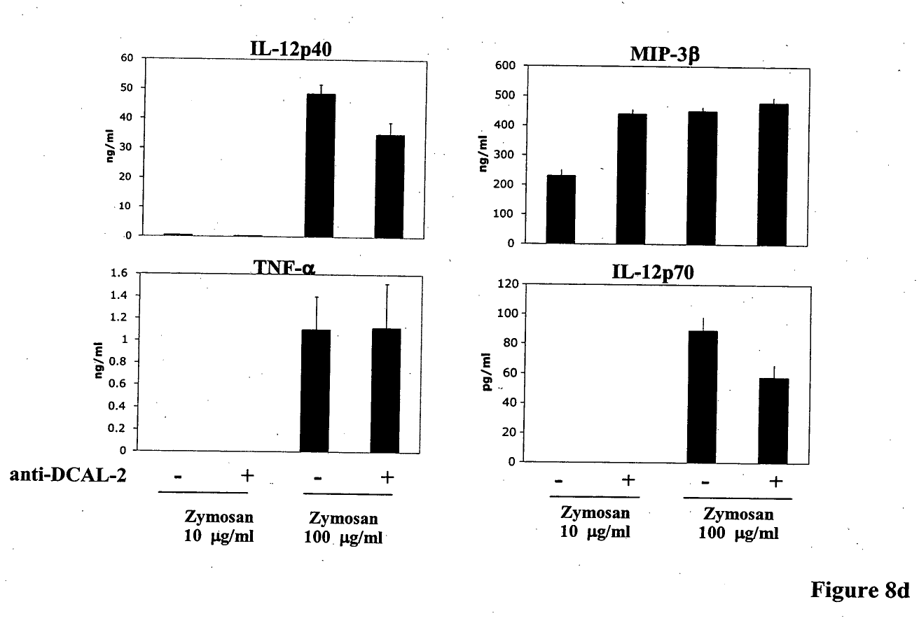

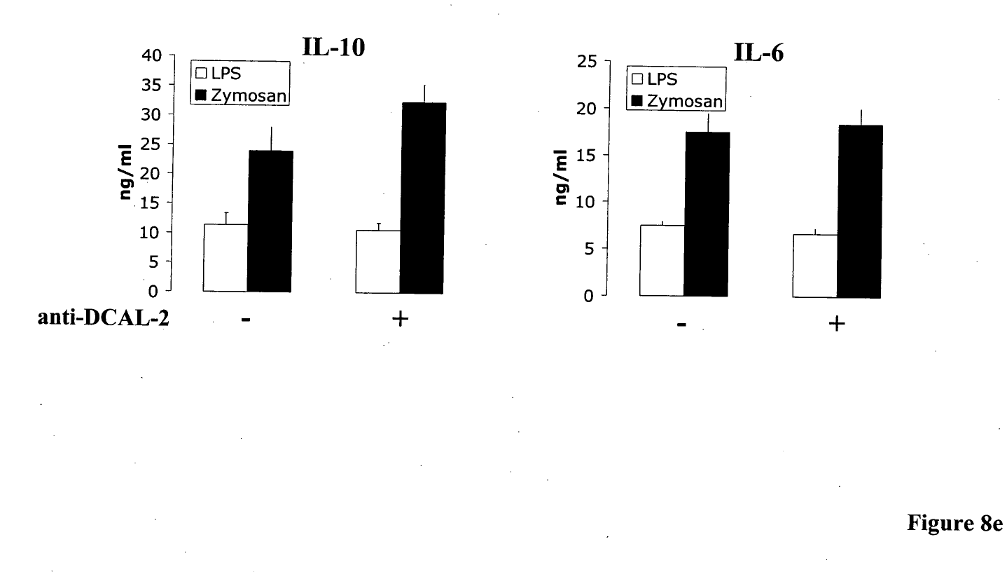

Upon stimulation, DCs express various cytokines and chemokines that are critical for directing innate and adaptive immune responses. Collaborations between TLRs and CLRs have been previously shown to modulate cytokine expression. We next examined if combining a DCAL-2 signal with different TLRs could modulate cytokines and chemokines expression patterns. To obtain a general pattern of DC cytokine/chemokine expression in response to LPS and LPS combined with anti-DCAL-2, we first applied DC culture supernatants from different stimuli to cytokine/chemokine protein arrays. Immature DCs express low level of chemokines including PARC, TARC, MDC, MCP-4 and Eotaxin-2 (FIG. 8a) and IL-8 and IL-12p40 (FIG. 8a). After stimulation with LPS, iDCs significantly increase expression cytokines including IL-10, IL-6, IL-1β, TNF-α IL-12 p40 and chemokines like MIP-1/3α, MIP-1β and RANTES. Surprisingly, stimulation with anti-DCAL-2 alone also induced increases in several cytokines and chemokines, including IL-6, IL-10 and IL-12p40 etc. similar to LPS stimulation, but also I-309 (CCL-1), MCP-2, TARC and MIP-3β. When DCs were stimulated with LPS and anti-DCAL-2, decreased levels of LPS induced IL-1β, TNF-α and MIP-3a were observed. The presence of LPS did not affect DCAL-2 induced 1-309 and MCP-2 expression.

A more quantitative analysis was then performed in which iDCs were stimulated with LPS and zymosan with or without anti-DCAL-2 mAb for 24 hours and culture supernatants harvested and analysed by ELISA to measure the levels of IL-12p40, IL-12p70, TNF-α, MIP-3β, IL-6 and IL-10 (FIGS. 8b-8e). As observed using cytokine arrays, anti-DCAL-2 stimulation alone induced moderate levels of IL-12p40, IL-6, IL-10, MIP-3β and lowered TNF-α expression (FIG. 8b). LPS and zymosan both induced significant amount of IL-12p40, IL-6, IL-10 and TNF-α. IL-12p70 was produced at low but detectable levels in the media from cells stimulated with LPS but not from those stimulated with zymosan or anti-DCAL-2 even though more IL-12p40 was detected. In the presence of DCAL-2 signals, LPS-induced IL-12p40, IL-12p70 and TNF-α expression was suppressed. Zymosan-induced IL-12p40 but not TNF-α expression was also suppressed by DCAL-2 signaling. Increasing level of MIP-3β could be observed when combining DCAL-2 and LPS signals, but not with the combination of DCAL-2 and Zymosan signals (FIGS. 8c and 8d). DCAL-2 signaling did not affect IL-6 and IL-10 expression by either LPS- or Zymosan-stimulated DCs (FIG. 8e). These data suggested that ligation of DCAL-2 could induce iDC to produce certain cytokines and chemokines, also DCAL-2 signal could antagonize TLR signals and selectively modulate TLR-induced cytokines/chemokines expression.

Enhanced Inflammatory Cytokine Expression DCAL-2 Signal Synergies with CD40 Pathway.

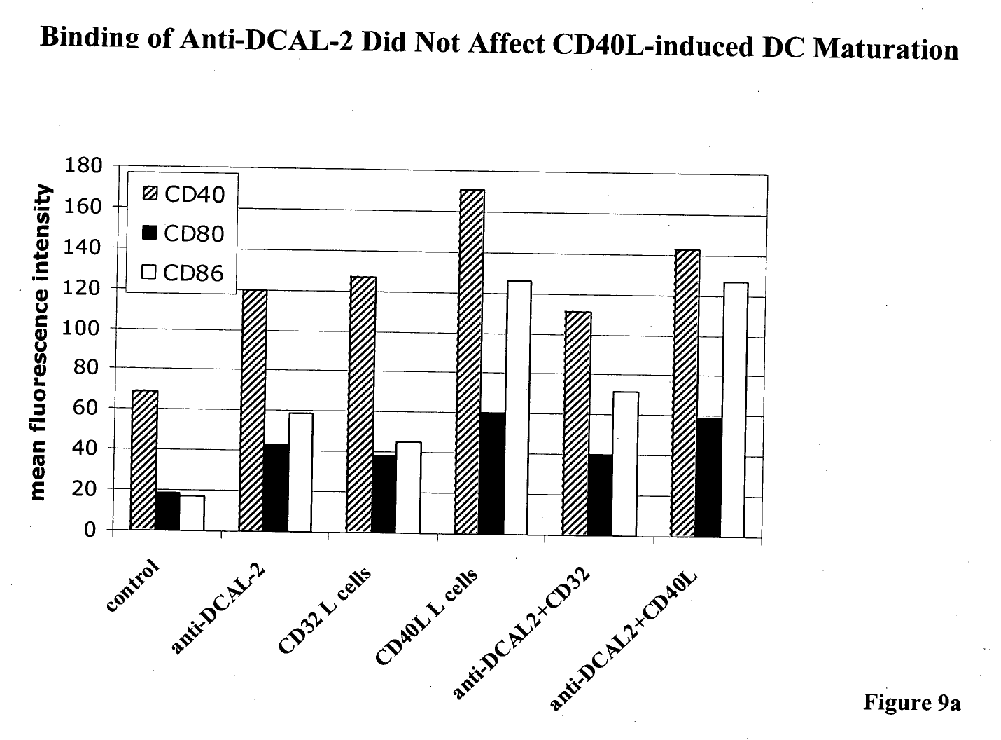

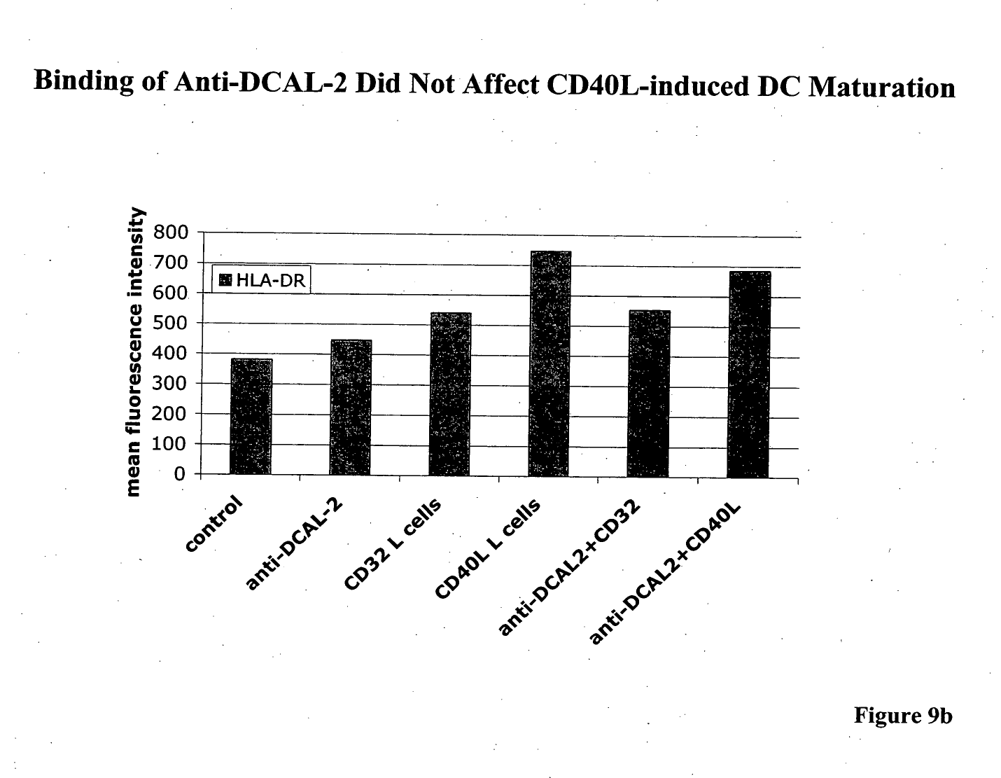

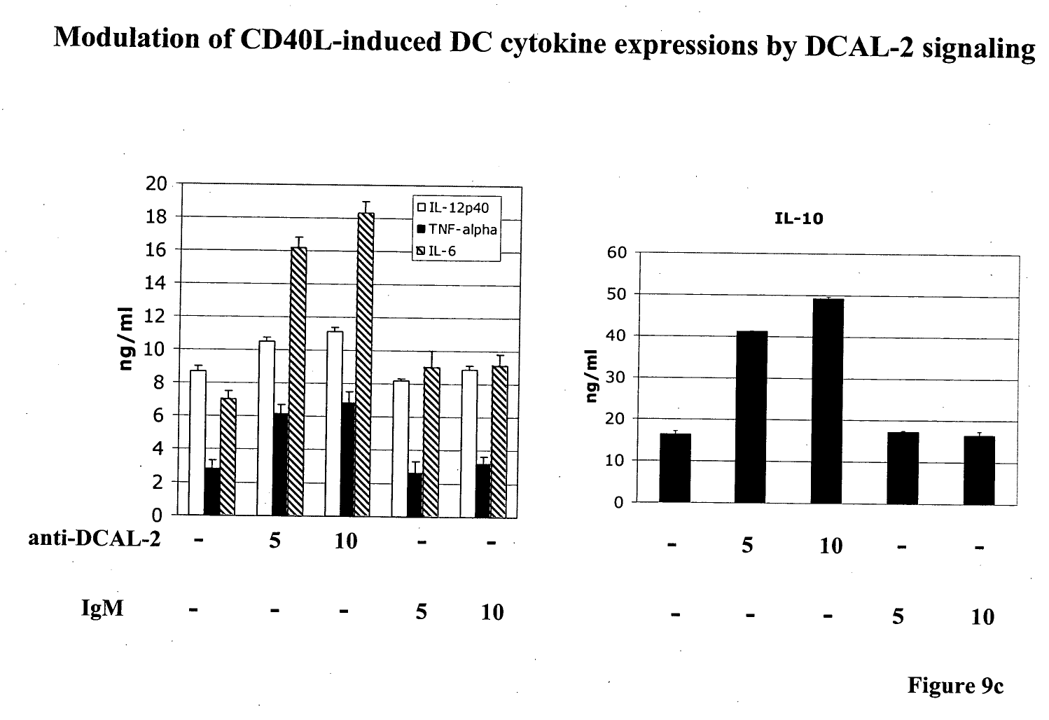

CD40 ligation of iDCs is one of the most potent stimuli for immature DCs. Since DCAL-2 ligation showed to modulate TLRs-induced DC functions, we examined whether DCAL-2 signaling could also interact with CD40 pathway. Cell surface marker analysis suggested that DCAL-2 did not affect CD86, CD80, CD40 and MHC class II expression (FIGS. 9a and 9b). Cytokine array analysis suggested that the levels of IL-1β and TNF-α were significantly increased by providing DCAL-2 signals (data not shown). ELISA analysis was used to further confirm the array data. The levels of TNF-α, IL-12p40, IL-12 p70, IL-6, IL-10 and MIP-3β were all increased with dose response when combining CD40 and DCAL-2 signaling together (FIG. 9c). Taken together, these results indicate that DCAL-2 signal can synergize with CD40 signal to promote inflammatory cytokine expression, and thus potentiate Th1 response.

### DCAL-2 Signaling Modulates the Capacity of DCs to Induce T Cell Activities

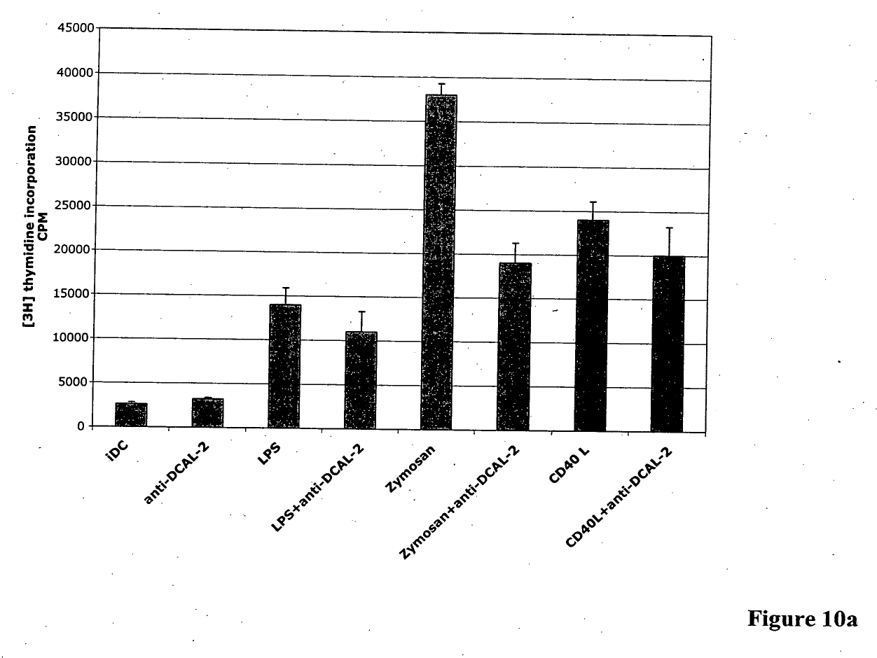

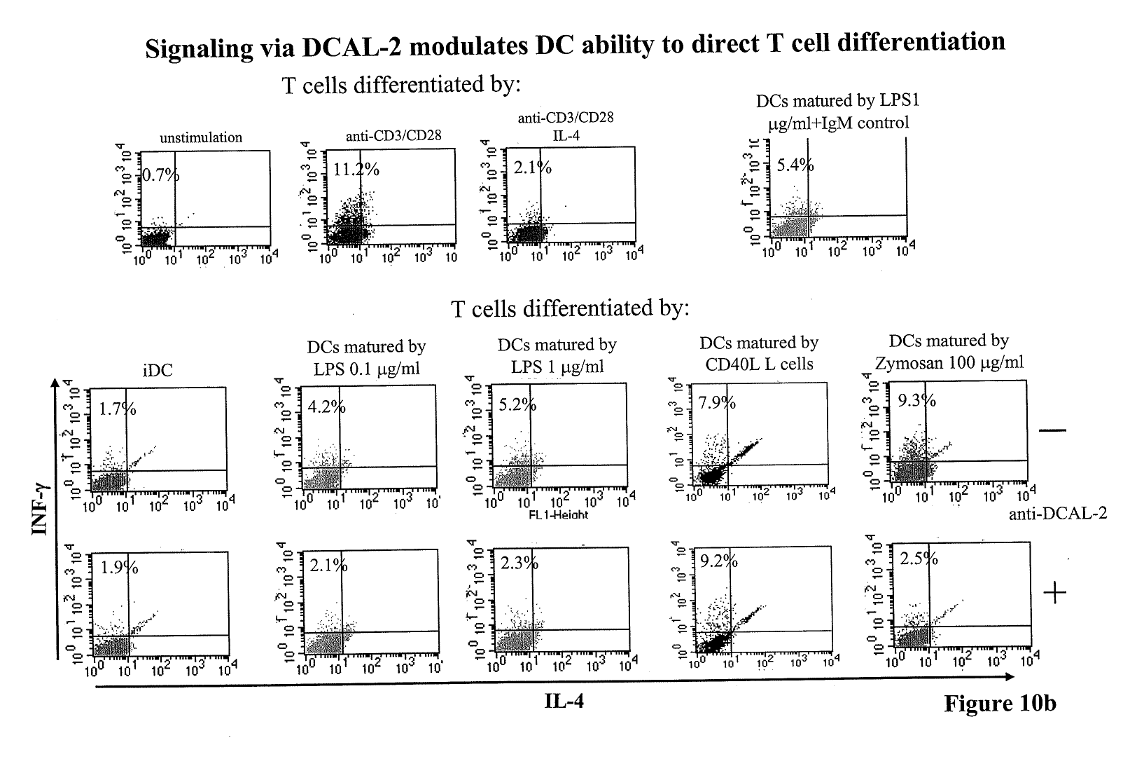

One important function of mature DCs is to activate resting T cells and induce cell proliferation and differentiation. We first used a mixed leukocyte response assay (a standard assay for measuring allogeneic T cell proliferation) to examine if DCAL-2 signaling would affect the capacity of DC to induce T cells proliferation. Monocyte-derived DCs were stimulated with LPS, Zymosan and CD40L with or without anti-DCAL-2 for 24 hours, then mixed with allogeneic CD4CD45RA T cells for 5 days. The results showed that DCAL-2 signal significantly decreased the capacity of Zymosan-matured DCs to induce naïve T cell proliferation, but not those matured by LPS or CD40L (FIG. 10a).

The balance of cytokine production (IL-12 vs IL-6/10) has been shown to be important for directing T cell differentiation. We next investigated if the reduced level of IL-12 expression in LPS/DCAL-2 and Zymosan/DCAL-2 treated DCs would affect T cell differentiation. As shown in FIG. 10b, when naïve T cells were co-cultured with LPS or zymosan matured-DC, there about 5% (by LPS-matured DCs) and 9% (by Zymosan-matured DCs) of differentiated T cells that produced IFN-γ when re-stimulated with PMA and lonomycin after 5 days of differentiation. However, when LPS/DCAL-2 or Zymosan/DCAL-2 matured DCs were used to differentiate naïve T cells, only 2.2% of T cells produced IFN-γ after re-stimulation. On the other hand, an increasing ratio of IFN-γ producing T cells was detected when CD40L/DCAL-2 matured-DCs were cultured with T cells as compared to CD40L matured DCs. These data indicate that the imbalance of IL-12 vs IL-6/10 expression caused by the presence of DCAL-2 signaling during DC maturation can influence downstream T cell differentiation.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, accession number, and patent applications cited herein are hereby incorporated by reference in their entirety for all purposes.

human DCAL-2 polypeptide sequence,
Accession no. AAS00605

SEQ ID NO: 1

MSEEVTYADLQFQNSSEMEKIPEIGKFGEKAPPAPSHVWRPAALFLTLLC
LLLLIGLGVLASMFHVTLKIEMKKMNKLQNISEELQRNISLQLMSNMNIS
NKIRNLSTTLQTIATKLCRELYSKEQEHKCKPCPRRWIWHKDSCYFLSDD
VQTWQESKMACAAQNASLLKINNKNALEFIKSQSRSYDYWLGLSPEEDST
RGMRVDNIINSSAWVIRNAPDLNNMYCGYINRLYVQYYHCTYKQRMICEK
MANPVQLGSTYFREA

What is claimed is:

1. A method of enhancing an immune response in a subject, the method comprising administering a DCAL-2 agonist to the subject in an amount sufficient to enhance an immune response.

2. The method of claim 1, wherein the agonist is an antibody that specifically binds SEQ ID NO:1.

3. The method of claim 2, wherein the antibody is humanized.

4. The method of claim 2, wherein the antibody is an scFv.

5. The method of claim 2, wherein the antibody is UW70 or a chimeric or humanized UW70.

6. The method of claim 1, further comprising administration of a vaccine.

7. The method of claim 1, wherein the agonist is administered with a vaccine.

8. A method of enhancing an immune response in a subject, the method comprising treating dendritic cells ex vivo with a DCAL-2 agonist in an amount sufficient to stimulate dendritic cell activity; and
introducing the treated dendritic cells into the subject.

9. The method of claim 8, wherein the agonist is an antibody that specifically binds DCAL-2.

10. The method of claim 8, further comprising administering a cancer antigen to the dendritic cells.

11. A method of modulating an immune response in a subject, the method comprising administering an agent that blocks DCAL-2 activation.

12. The method of claim 11, wherein the agent is a monoclonal antibody.

13. The method of claim 12, wherein the monoclonal antibody is humanized.

14. The method of claim 11, wherein the agent is a soluble DCAL-2 receptor.

15. The method of claim 11, wherein the subject has an autoimmune disease selected from the group consisting of multiple sclerosis, psoriasis, rheumatoid arthritis, and insulin-dependent diabetes.

16. The method of claim 11, wherein the subject has asthma, an allergy, or a chronic inflammatory disease.

17. The method of claim 11, wherein the agent is administered in a subject that has a transplantation reaction for allogeneic or xenogeneic transplants or graft-vs. host disease in bone marrow transplants.

18. A method of augmenting a protective immune response, the method comprising administering a monoclonal that binds to SEQ ID NO:1 with a vaccine to a cancer antigen or pathogen, where the antibody is administered in an amount sufficient to augment the response to the antigen.


SEQUENCE LISTING (12 sequences)

SEQ ID NO: 1 - protein, 265 aa, Homo sapiens
  human dendritic cell associated lectin 2 (DCAL-2), myeloid inhibitory C-type lectin-like receptor isoform alpha (MICL-alpha), type II transmembrane protein, C-type lectin protein (CLL-1)
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKQRMICEKMANPVQLGSTYFREA

SEQ ID NO: 2 - DNA, 798 bp, Homo sapiens
  human dendritic cell associated lectin 2 (DCAL-2), myeloid inhibitory C-type lectin-like receptor isoform alpha (MICL-alpha), type II transmembrane protein, C-type lectin protein (CLL-1) cDNA
atgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcatgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgggttataagaaacgcacctgacttaaataacatgtattgtggatatataaatagactatatgttcaatattatcactgcacttataaacaaagaatgatatgtgagaagatggccaatccagtgcagcttggttctacatattttagggaggcatga

SEQ ID NO: 3 - DNA, 804 bp, Mus musculus
  mouse dendritic cell associated lectin 2 (DCAL-2), myeloid inhibitory C-type lectin-like receptor isoform alpha (MICL-alpha), type II transmembrane protein, C-type lectin protein (CLL-1)
atgtctgaagaaattgtttatgcaaatctcaaaatccaggaccctgataaaaaagaagaaacccagaagtctgacaaatgtgggggaaaagtatccgccgatgcttcccattcacagcaaaaaacagtcttgattctgattcttctatgccttctgctgttcattggaatgggggtcttaggaggcatcttttatacaactttggcaacagaaatgataaaatcgaatcaattgcaaagggccaaggaagaacttcaggaaaatgtttccctacagctgaagcacaatctcaacagctccaagaaaatcaagaacctttctgccatgctgcaaagcacagccacccagctgtgccgagagctgtatagcaaagaaccagagcacaaatgtaaaccatgtccaaagggttcagaatggtacaaggacagctgttattctcaactcaatcagtatggaacatggcaagagagtgtcatggcctgctctgctcggaatgccagcctcctgaaggttaagaacaaggatgtgctggaatttataaagtacaagaagctacgctatttttggcttgcattgttgcccagaaaagatcgcacacaatatccactaagtgagaagatgttcctctctgaagagtctgaaagaagcacagatgacatagataagaagtactgcggatatatagacagggtcaatgtttattatacatactgcactgatgagaacaatatcatatgtgaagagacagccagcaaggtgcagttggaaagtgtgttgaatggcctcccagaggatagcaggtag

SEQ ID NO: 4 - protein, 267 aa, Mus musculus
  mouse DCAL-2
MSEEIVYANLKIQDPDKKEETQKSDKCGGKVSADASHSQQKTVLILILLCLLLFIGMGVLGGIFYTTLATEMIKSNQLQRAKEELQENVSLQLKHNLNSSKKIKNLSAMLQSTATQLCRELYSKEPEHKCKPCPKGSEWYKDSCYSQLNQYGTWQESVMACSARNASLLKVKNKDVLEFIKYKKLRYFWLALLPRKDRTQYPLSEKMFLSEESERSTDDIDKKYCGYIDRVNVYYTYCTDENNIICEETASKVQLESVLNGLPEDSR

SEQ ID NO: 5 - protein, 4 aa, Artificial Sequence
  Description of Artificial Sequenceextension of hydrophobic transmembrane domain
LFLT

SEQ ID NO: 6 - DNA, 28 bp, Artificial Sequence
  Description of Artificial Sequencehuman DCAL-2 cDNA PCR specific primer
cattcagctctgttaactcactcatctt

SEQ ID NO: 7 - DNA, 28 bp, Artificial Sequence
  Description of Artificial Sequencehuman DCAL-2 cDNA PCR specific primer
cacctattatattagttgaggagacgga

SEQ ID NO: 8 - DNA, 26 bp, Artificial Sequence
  Description of Artificial Sequencebeta-actin loading control forward primer
gtcgtcgacaacggctccggcatctg

SEQ ID NO: 9 - DNA, 26 bp, Artificial Sequence
  Description of Artificial Sequencebeta-actin loading control reverse primer
ctagaccgtggtgtggaagatgttac

SEQ ID NO: 10 - DNA, 30 bp, Artificial Sequence
  Description of Artificial SequenceDCAL-2 extracellular region cloning primer DCAL-2 (BamH I)
gttgttggatccggcaagcatgtttcatgt

SEQ ID NO: 11 - DNA, 31 bp, Artificial Sequence
  Description of Artificial SequenceDCAL-2 extracellular region cloning primer (HindIII)
cgcaagctttgttgcctccctaaaatatgta

SEQ ID NO: 12 - protein, 6 aa, Artificial Sequence
  Description of Artificial Sequencetypical type II transmembrane C-type lectin cytoplasmic tail, immunoreceptor tyrosine-based inhibitory motif (ITIM)
VTYADL